(19) United States
(12) Patent Application Publication
(10) Pub. No.: US 2017/0028013 A1
(43) Pub. Date: Feb. 2, 2017

(54) COMBINATION FORMULATION OF LAQUINIMOD AND GLATIRAMER ACETATE WITH AMINO ACIDS

(71) Applicants: Galina Linden, Bat-Yam (IL); Rakefet Cohen, Kokhave Yair - Zur Yga'al (IL); Danit Licht, Givat Shmuel (IL)

(72) Inventors: Galina Linden, Bat-Yam (IL); Rakefet Cohen, Kokhave Yair - Zur Yga'al (IL); Danit Licht, Givat Shmuel (IL)

(73) Assignee: Teva Pharmaceutical Industries, Ltd., Petach-Tikva (IL)

(21) Appl. No.: 15/222,213

(22) Filed: Jul. 28, 2016

Related U.S. Application Data
(60) Provisional application No. 62/203,814, filed on Aug. 11, 2015.
(60) Provisional application No. 62/198,845, filed on Jul. 30, 2015.

(51) Int. Cl. A61K 38/02; A61K 47/02; A61K 9/00; A61K 47/26; A61K 31/4704; A61K 31/198

(52) U.S. Cl. CPC A61K 38/02; A61K 31/4704; A61K 31/198; A61K 9/0053; A61K 47/26; A61K 47/02

(57) ABSTRACT

A stable pharmaceutical composition comprising a therapeutically effective amount of laquinimod, a therapeutically effective amount of glatiramer acetate related drug substance, and an amount of an amino acid. A process for making a stable pharmaceutical in liquid form, a sealed package comprising a stable pharmaceutical composition as described herein, and a use of a stable pharmaceutical composition as described herein. A method for treating a subject afflicted with multiple sclerosis or presenting a clinically isolated syndrome, alleviating a symptom of multiple sclerosis in a subject afflicted with multiple sclerosis or presenting a clinically isolated syndrome, and/or providing neuroprotection to a subject in need thereof, comprising administering to the subject a composition as described herein.

This application claims the benefit of U.S. Provisional Application No. 62/198,845, filed Jul. 30, 2015, and U.S. Provisional Application No. 62/203,814, filed Aug. 11, 2015, the entire contents of each of which are hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND

### Laquinimod

Laquinimod is a compound which has been shown to be effective in the acute experimental autoimmune encephalomyelitis (aEAE) model (U.S. Pat. No. 6,077,851). Its chemical name is N-ethyl-N-phenyl-1,2-dihydro-4-hydroxy-5-chloro-1-methyl-2-oxoquinoline-3-carboxamide, and its Chemical Registry number is 248281-84-7. The processes of synthesis of laquinimod and the preparation of its sodium salt are disclosed in U.S. Pat. No. 6,077,851. An additional process of synthesis of laquinimod is disclosed in U.S. Pat. No. 6,875,869.

Pharmaceutical compositions comprising laquinimod sodium are disclosed in, e.g., U.S. Pat. No. 7,989,473 and PCT International Application Publication No. WO 2005/074899.

Laquinimod sodium has high oral bioavailability and has been suggested as an oral formulation for the treatment of Multiple Sclerosis (MS). (Polman, 2005 and Sandberg-Wollheim, 2005). Studies have also shown that laquinimod can reduce development of active MRI lesions in relapsing MS. (Polman 2005).

### Glatiramer Acetate

Glatiramer acetate (GA), also known as Copolymer-1, has been shown to be effective in treating multiple sclerosis (MS) (Lampert, 1978). Daily subcutaneous injections of glatiramer acetate (20 mg/injection) reduce relapse rates, progression of disability, appearance of new lesions by magnetic resonance imaging (MRI), (Johnson, 1995) and appearance of “black holes” (Filippi, 2001).

COPAXONE® is the brand name for a formulation containing glatiramer acetate as the active ingredient. Glatiramer acetate is approved for reducing the frequency of relapses in relapsing-remitting multiple sclerosis (RRMS). Glatiramer acetate consists of the acetate salts of synthetic polypeptides containing four naturally occurring amino acids: L-glutamic acid, L-alanine, L-tyrosine, and L-lysine with an average molar fraction in COPAXONE® of 0.141, 0.427, 0.095 and 0.338, respectively. In COPAXONE®, the average molecular weight of the glatiramer acetate is 4,700-11,000 daltons. Chemically, glatiramer acetate is designated L-glutamic acid polymer with L-alanine, L-lysine and L-tyrosine, acetate (salt). Its structural formula is:

(Glu,Ala,Lys,Tyr)xACH3COOH

(C5H9NO4AC3H7NO2AC6H14N2O2AC9H11NO3)PAPC2H4O2

CAS—147245-92-9.

The recommended dosing schedule of COPAXONE® for relapsing-remitting multiple sclerosis is 20 mg per day or 40 mg three times per week injected subcutaneously (Physician's Desk Reference; see also U.S. Pat. Nos. 3,849,550; 5,800,808; 5,858,964, 5,981,589; 6,048,898; 6,054,430; 6,214,791; 6,342,476; 6,362,161; and 8,399,413, all of which are hereby incorporated by reference).

### Combination of Laquinimod and Glatiramer Acetate

Laquinimod as an add-on therapy to or in combination with GA for treating multiple sclerosis was described in U.S. Application Publication No. 2013-0029916. Stable formulations comprising laquinimod and GA have not been reported.

### SUMMARY OF THE INVENTION

The subject invention provides a stable pharmaceutical composition comprising a therapeutically effective amount of laquinimod, a therapeutically effective amount of glatiramer acetate related drug substance, and an amount of an amino acid.

The subject invention also provides a composition comprising a therapeutically effective amount of laquinimod and an amount of an amino acid.

The subject invention also provides a composition comprising a therapeutically effective amount of glatiramer acetate related drug substance and an amount of an amino acid.

The subject invention also provides a process for making a stable pharmaceutical liquid form comprising a therapeutically effective amount of laquinimod or pharmaceutically acceptable salt thereof, a therapeutically effective amount of glatiramer acetate related drug substance, and an amount of an amino acid comprising: a) obtaining the laquinimod or pharmaceutically acceptable salt thereof, the glatiramer acetate related drug substance, and the amino acid, and b) forming the liquid form of the laquinimod or pharmaceutically acceptable salt thereof, the glatiramer acetate related drug substance, and the amino acid.

The subject invention also provides a process for making a liquid form comprising a therapeutically effective amount of glatiramer acetate related drug substance and an amount of an amino acid comprising: a) obtaining the glatiramer acetate related drug substance and the amino acid, and b) forming the liquid form of the glatiramer acetate related drug substance and the amino acid.

The subject invention also provides a process for making a liquid form comprising a therapeutically effective amount of laquinimod or pharmaceutically acceptable salt thereof and an amount of an amino acid comprising: a) obtaining the laquinimod or pharmaceutically acceptable salt thereof and the amino acid, and b) forming the liquid form of the laquinimod or pharmaceutically acceptable salt thereof and the amino acid.

The subject invention also provides a stable pharmaceutical composition comprising a therapeutically effective amount of laquinimod or pharmaceutically acceptable salt thereof, a therapeutically effective amount of glatiramer acetate related drug substance, and an amount of an amino acid, prepared by the processes described herein.

The subject invention also provides a sealed package comprising the compositions described herein.

The subject invention also provides a method for treating a subject afflicted with a form of autoimmune disease comprising administering to the subject a composition as described herein so as to thereby treat the subject.

In one embodiment, the autoimmune disease is multiple sclerosis. In one embodiment, the multiple sclerosis is relapsing multiple sclerosis. In one embodiment, the relapsing multiple sclerosis is relapsing-remitting multiple sclerosis.

The subject invention also provides a method for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome, comprising administering to the subject a composition as described herein so as to thereby treat and/or alleviate the symptom of multiple sclerosis in the subject.

The subject invention also provides a method for providing neuroprotection to a subject in need thereof comprising administering to the subject a composition as described herein so as to thereby provide neuroprotection to the subject.

The subject invention also provides use of a composition as described herein for treating a subject afflicted with an autoimmune disease.

The subject invention also provides use of a composition as described herein for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome.

The subject invention also provides use of a composition as described herein for providing neuroprotection to a subject in need thereof.

The subject invention also provides use of a composition as described herein in the manufacture of a medicament for treating a subject afflicted with an autoimmune disease.

The subject invention also provides use of a composition as described herein in the manufacture of a medicament for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome.

The subject invention also provides use of a composition as described herein in the manufacture of a medicament for providing neuroprotection to a subject in need thereof.

The subject invention also provides a composition as described herein for use in a) treating a subject afflicted with an autoimmune disease, b) i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome, and/or c) providing neuroprotection to a subject in need thereof.

The subject invention also provides a pharmaceutical oral unit dosage form of a therapeutically effective amount of laquinimod or pharmaceutically acceptable salt thereof, a therapeutically effective amount of glatiramer acetate related drug substance, and an amount of an amino acid.

The subject invention also provides a package comprising: a) one or more unit doses, each such unit dose comprising i) a therapeutically effective amount of laquinimod or pharmaceutically acceptable salt thereof, ii) a therapeutically effective amount of glatiramer acetate related drug substance, and iii) an amount of an amino acid, and b) instructions for use of the one or more unit doses for i) treating a subject afflicted with an autoimmune disease, ii) treating a subject afflicted with multiple sclerosis or presenting a clinically isolated syndrome, and/or alleviating a symptom of multiple sclerosis in a subject afflicted with multiple sclerosis or presenting a clinically isolated syndrome, and/or iii) providing neuroprotection to a subject in need thereof.

### BRIEF DESCRIPTION OF THE DRAWINGS

FIG. 1 is an illustration of diverse nanoparticles aimed to be used in drug delivery of nanodrugs.

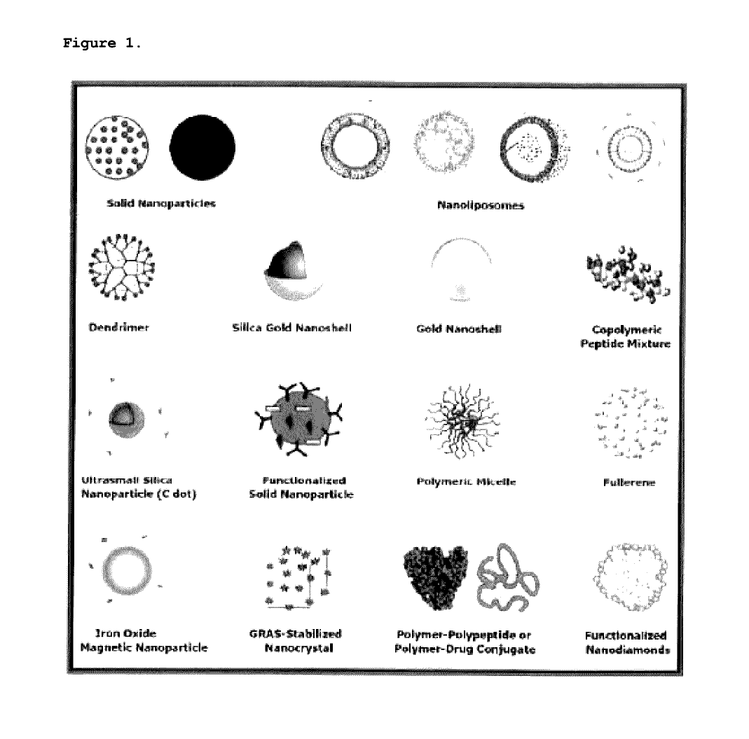

FIG. 2 is an illustration of the Ubbelohde Viscometer.

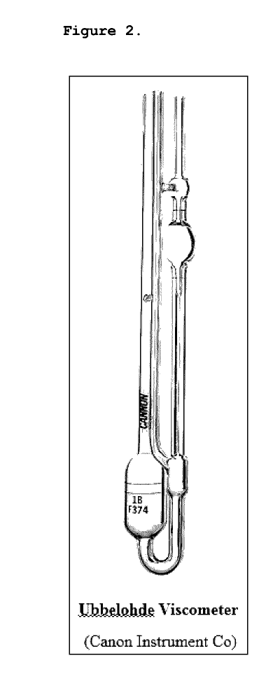

FIG. 3 shows the gelation (on the y-axis) in relation to the concentration of L-lysine (mg/mL) (on the x-axis).

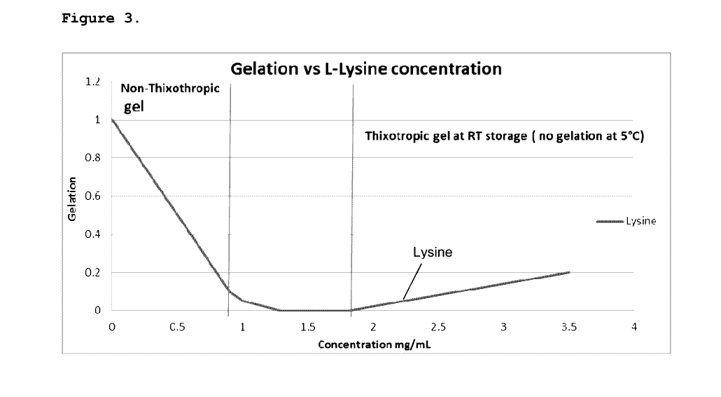

FIGS. 4A and 48 show the gelation (on the y-axis) in relation to the concentration of amino acids glycine, lysine, proline, histidine, and alanine (mg/mL) (on the x-axis).

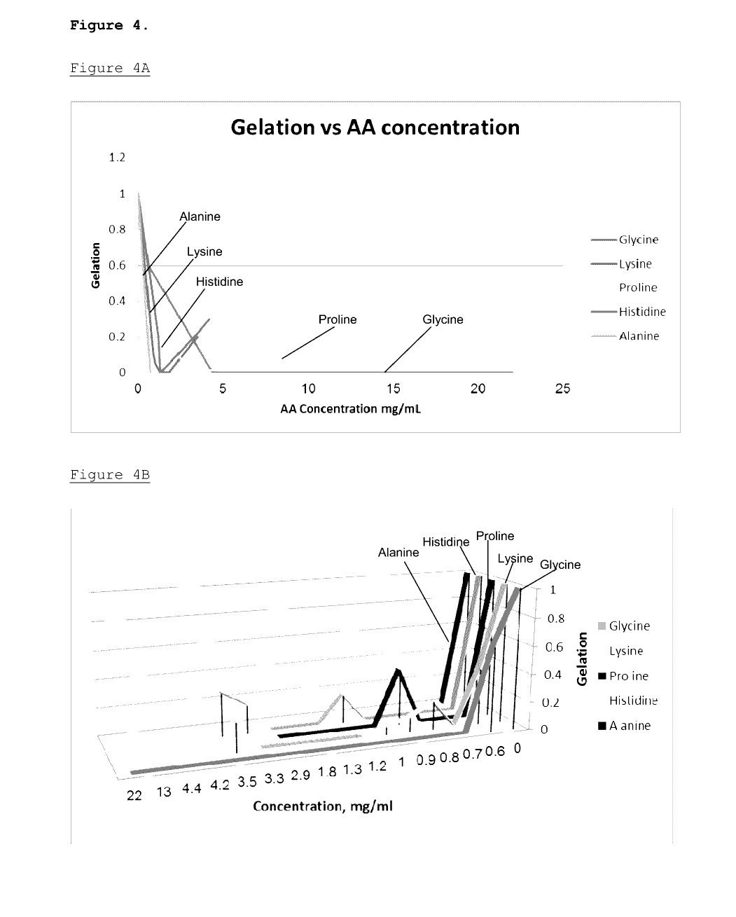

FIG. 5 shows the least squares analysis of the gelation of GA-LAQ formulation with L-lysine and pure water (actual gelation on the y-axis; gelation predicted on the x-axis).

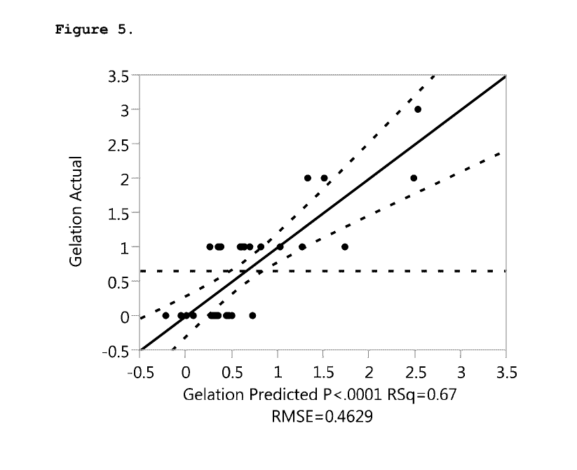

FIG. 6 is a laquinimod leverage plot with gelation leverage residuals on the y-axis and laquinimod leverage on the x-axis.

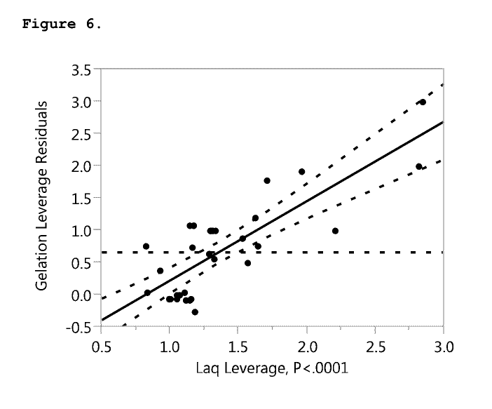

FIG. 7 is a GA leverage plot with gelation leverage residuals on the y-axis and GA leverage on the x-axis.

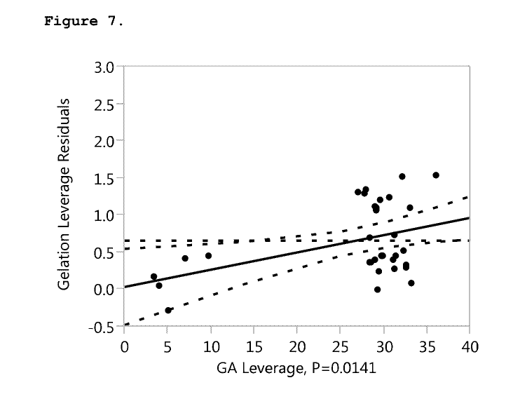

### DETAILED DESCRIPTION OF THE INVENTION

The subject invention provides a stable pharmaceutical composition comprising a therapeutically effective amount of laquinimod, a therapeutically effective an amount of glatiramer acetate related drug substance, and an amount of an amino acid.

The subject invention also provides a composition comprising a therapeutically effective amount of laquinimod and an amount of an amino acid.

The subject invention also provides a composition comprising a therapeutically effective amount of glatiramer acetate related drug substance and an amount of an amino acid.

In one embodiment, the composition is in liquid form. In one embodiment, the composition is a lyophilized powder. In one embodiment, the composition is an aqueous solution. In one embodiment, the composition is clear. In one embodiment, the composition is isotonic.

In one embodiment, the laquinimod is a pharmaceutically acceptable salt of laquinimod. In another embodiment, the pharmaceutically acceptable salt of laquinimod is potassium salt, lithium salt, sodium salt or calcium salt. In another embodiment, the pharmaceutically acceptable salt of laquinimod is laquinimod sodium.

In one embodiment, the laquinimod is laquinimod acid.

In one embodiment, the therapeutically effective amount of laquinimod is less than 0.6 mg. In another embodiment, the therapeutically effective amount of laquinimod is 0.1-40.0 mg. In another embodiment, the therapeutically effective amount of laquinimod is 0.25 mg-1.5 mg. In another embodiment, the therapeutically effective amount of laquinimod is 0.1 mg. In another embodiment, the therapeutically effective amount of laquinimod is 0.25 mg. In another embodiment, the therapeutically effective amount of laquinimod is 0.3 mg. In another embodiment, the therapeutically effective amount of laquinimod is 0.5 mg. In another embodiment, the therapeutically effective amount of laquinimod is 0.6 mg. In another embodiment, the therapeutically effective amount of laquinimod is 1.0 mg. In yet another embodiment, the therapeutically effective amount of laquinimod is 1.2 mg. In another embodiment, the therapeutically effective amount of laquinimod is 1.5 mg. In another embodiment, the therapeutically effective amount of laquinimod is 2.0 mg.

In one embodiment, the therapeutically effective amount of laquinimod has a concentration of 0.1-3 mg/mL. In one embodiment, the therapeutically effective amount of laquinimod has a concentration of 0.7 mg/mL. In one embodiment, the therapeutically effective amount of laquinimod has a concentration of 0.8 mg/mL. In one embodiment, the therapeutically effective amount of laquinimod has a concentration of 0.9 mg/mL. In one embodiment, the therapeutically effective amount of laquinimod has a concentration of 1 mg/mL. In one embodiment, the therapeutically effective amount of laquinimod has a concentration of 1.1 mg/mL. In one embodiment, the therapeutically effective amount of laquinimod has a concentration of 1.25 mg/mL. In one embodiment, the therapeutically effective amount of laquinimod has a concentration of 1.5 mg/mL. In one embodiment, the therapeutically effective amount of laquinimod has a concentration of 2 mg/mL. In one embodiment, the therapeutically effective amount of laquinimod has a concentration of 3 mg/mL.

In one embodiment, the glatiramer acetate related drug substance is glatiramer acetate.

In one embodiment, the therapeutically effective amount of glatiramer acetate related drug substance is 0.1-1000 mg. In one embodiment, the therapeutically effective amount of glatiramer acetate related drug substance is 50-150 mg. In one embodiment, the therapeutically effective amount of glatiramer acetate related drug substance is 0.1-70 mg. In one embodiment, the therapeutically effective amount of glatiramer acetate related drug substance is 10-80 mg. In one embodiment, the therapeutically effective amount of glatiramer acetate related drug substance is 1 mg. In one embodiment, the therapeutically effective amount of glatiramer acetate related drug substance is 5 mg. In one embodiment, the therapeutically effective amount of glatiramer acetate related drug substance is 15 mg. In one embodiment, the therapeutically effective amount of glatiramer acetate related drug substance is 20 mg. In one embodiment, the therapeutically effective amount of glatiramer acetate related drug substance is 30 mg. In one embodiment, the therapeutically effective amount of glatiramer acetate related drug substance is 40 mg. In one embodiment, the therapeutically effective amount of glatiramer acetate related drug substance is 50 mg. In one embodiment, the therapeutically effective amount of glatiramer acetate related drug substance is 100 mg.

In one embodiment, the therapeutically effective amount of glatiramer acetate related drug substance has a concentration of 5-40 mg/mL. In one embodiment, the therapeutically effective amount of glatiramer acetate related drug substance has a concentration of 5 mg/mL. In one embodiment, the therapeutically effective amount of glatiramer acetate related drug substance has a concentration of 10 mg/mL. In one embodiment, the therapeutically effective amount of glatiramer acetate related drug substance has a concentration of 30 mg/mL. In one embodiment, the therapeutically effective amount of glatiramer acetate related drug substance has a concentration of 35 mg/mL. In one embodiment, the therapeutically effective amount of glatiramer acetate related drug substance has a concentration of 40 mg/mL.

In one embodiment, the composition is in a unit dose. In one embodiment, the unit dose comprises 0.1 mg, 0.25 mg, 0.3 mg, 0.5 mg, 0.6 mg, 1.0 mg, 1.2 mg, 1.5 mg, or 2.0 mg of laquinimod and 1 mg, 5 mg, 15 mg, 20 mg, 30 mg, 40 mg, 50 mg, or 100 mg of glatiramer acetate related drug substance.

In one embodiment, the unit dose has a volume of 0.5-5 mL. In one embodiment, the unit dose has a volume of 1.0 mL. In another embodiment, the unit dose has a volume of 1.5 mL. In another embodiment, the unit dose has a volume of 2 mL. In another embodiment, the unit dose has a volume of 3 mL. In another embodiment, the unit dose has a volume of 4 mL. In another embodiment, the unit dose has a volume of 5 mL.

In one embodiment, the unit dose is in a syringe. In one embodiment, the unit dose is in a pre-filled syringe for administration. In another embodiment, the unit dose is in a vial. In another embodiment, the unit dose is in an ampule. In another embodiment, the unit dose is in a cartridge. In another embodiment, the unit dose is in an infusion.

In one embodiment, the amino acid is selected from lysine, glycine, proline, alanine, or histidine.

In one embodiment, the amino acid is lysine. In one embodiment, the amino acid is glycine. In one embodiment, the amino acid is proline. In one embodiment, the amino acid is alanine. In one embodiment, the amino acid is histidine.

In one embodiment, the amino acid has a concentration of 0.5-22 mg/mL. In one embodiment, the amino acid has a concentration of 0.5-5 mg/mL. In one embodiment, the amino acid has a concentration of 0.5-3.5 mg/mL. In one embodiment, the amino acid has a concentration of 0.7-1.8 mg/mL. In one embodiment, the amino acid has a concentration of 0.5 mg/mL. In another embodiment, the amino acid has a concentration of 0.6 mg/mL. In another embodiment, the amino acid has a concentration of 0.7 mg/mL. In another embodiment, the amino acid has a concentration of 0.8 mg/mL. In another embodiment, the amino acid has a concentration of 0.9 mg/mL. In another embodiment, the amino acid has a concentration of 1 mg/mL. In another embodiment, the amino acid has a concentration of 1.1 mg/mL. In another embodiment, the amino acid has a concentration of 1.2 mg/mL. In another embodiment, the amino acid has a concentration of 1.3 mg/mL. In another embodiment, the amino acid has a concentration of 1.4 mg/mL. In another embodiment, the amino acid has a concentration of 1.5 mg/mL. In another embodiment, the amino acid has a concentration of 2 mg/mL. In another embodiment, the amino acid has a concentration of 2.25 mg/mL. In another embodiment, the amino acid has a concentration of 2.5 mg/mL. In another embodiment, the amino acid has a concentration of 3 mg/mL. In another embodiment, the amino acid has a concentration of 3.5 mg/mL. In another embodiment, the amino acid has a concentration of 4 mg/mL. In another embodiment, the amino acid has a concentration of 5 mg/mL.

In one embodiment, the concentration ratio of lysine to laquinimod (Lys:Laq) is from about 0.4 mg/mL:1 mg/mL to about 3.2 mg/mL:1 mg/mL.

In one embodiment, the concentration ratio of glycine to laquinimod (Gly:Laq) is from about 0.5 mg/mL:1 mg/mL to about 4.1 mg/mL:1 mg/mL.

In one embodiment, the concentration ratio of proline to laquinimod (Pro:Laq) is from about 1.5 mg/mL:1 mg/mL to about 2.2 mg/mL:1 mg/mL.

In one embodiment, the concentration ratio of alanine to laquinimod (Ala:Laq) is from about 0.6 mg/mL:1 mg/mL to about 0.7 mg/mL:1 mg/mL.

In one embodiment, the concentration ratio of histidine to laquinimod (His:Laq) is from about 1.2 mg/mL:1 mg/mL to about 2.6 mg/mL:1 mg/mL.

In one embodiment, the composition further comprises a tonicity agent. In one embodiment, the tonicity agent is mannitol, sodium chloride, or trehalose.

In one embodiment, the tonicity agent is mannitol. In one embodiment, the mannitol has a concentration of 7-50 mg/mL. In one embodiment, the mannitol has a concentration of 7 mg/mL. In one embodiment, the mannitol has a concentration of 20 mg/mL. In one embodiment, the mannitol has a concentration of 25 mg/mL. In one embodiment, the mannitol has a concentration of 30 mg/mL. In one embodiment, the mannitol has a concentration of 35 mg/mL. In one embodiment, the mannitol has a concentration of 37.5 mg/mL. In one embodiment, the mannitol has a concentration of 40 mg/mL. In one embodiment, the mannitol has a concentration of 43 mg/mL. In one embodiment, the mannitol has a concentration of 45 mg/mL. In one embodiment, the mannitol has a concentration of 50 mg/mL.

In one embodiment, the tonicity agent is sodium chloride. In one embodiment, the sodium chloride has a concentration of 5-10 mg/mL. In one embodiment, the sodium chloride has a concentration of 5 mg/mL. In one embodiment, the sodium chloride has a concentration of 6 mg/mL. In one embodiment, the sodium chloride has a concentration of 7 mg/mL. In one embodiment, the sodium chloride has a concentration of 8 mg/mL. In one embodiment, the sodium chloride has a concentration of 9 mg/mL. In one embodiment, the sodium chloride has a concentration of 10 mg/mL.

In one embodiment, the composition further comprises a buffer. In one embodiment, the buffer is histidine, phosphate-buffered saline, phosphate salt, hydrocarbonate, or acetate. In one embodiment, the phosphate salt is a sodium salt, potassium salt, or sodium-potassium salt. In one embodiment, the hydrocarbonate is sodium bicarbonate. In one embodiment, the acetate is sodium acetate.

In one embodiment, the buffer has a concentration of 0.1-0.5 mol/L. In one embodiment, the buffer has a concentration of 0.1 mol/L. In another embodiment, the buffer has a concentration of 0.5 mol/L.

In one embodiment, the composition is prepared for periodic administration. In one embodiment, the periodic administration continues for more than 30 days. In another embodiment, the periodic administration continues for more than 42 days. In another embodiment, the periodic administration continues for 6 months or more.

In one embodiment, the composition is prepared for administration once daily. In another embodiment, the composition is prepared for administration more often than once daily. In one embodiment, the composition is prepared for administration three times per week. In another embodiment, the composition is prepared for administration less often than once daily.

In one embodiment, the composition is prepared for administration by subcutaneous injection. In another embodiment, the composition is prepared for administration through an intravenous, intraperitoneal, intramuscular, intranasal, buccal, vaginal, rectal, intraocular, intrathecal, topical, oral, or intradermal route.

The subject invention also provides a process for making a stable pharmaceutical liquid form comprising a therapeutically effective amount of laquinimod or pharmaceutically acceptable salt thereof, a therapeutically effective amount of glatiramer acetate related drug substance, and an amount of an amino acid comprising: a) obtaining the laquinimod or pharmaceutically acceptable salt thereof, the glatiramer acetate related drug substance, and the amino acid, and b) forming the liquid form of the laquinimod or pharmaceutically acceptable salt thereof, the glatiramer acetate related drug substance, and the amino acid.

The subject invention also provides a process for making a liquid form comprising a therapeutically effective amount of glatiramer acetate related drug substance and an amount of an amino acid comprising: a) obtaining the glatiramer acetate related drug substance and the amino acid, and b) forming the liquid form of the glatiramer acetate related drug substance and the amino acid.

In one embodiment, the glatiramer acetate related drug substance is glatiramer acetate.

The subject invention also provides a process for making a liquid form comprising a therapeutically effective amount of laquinimod or pharmaceutically acceptable salt thereof and an amount of an amino acid comprising: a) obtaining the laquinimod or pharmaceutically acceptable salt thereof and the amino acid, and b) forming the liquid form of the laquinimod or pharmaceutically acceptable salt thereof and the amino acid.

In one embodiment, the process comprises forming a liquid form of the glatiramer acetate related drug substance and the amino acid prior to forming the liquid form of the laquinimod or pharmaceutically acceptable salt thereof, the glatiramer acetate related drug substance, and the amino acid.

In one embodiment, the process comprises forming a liquid form of the laquinimod or pharmaceutically acceptable salt thereof and the amino acid prior to forming the liquid form of the laquinimod or pharmaceutically acceptable salt thereof, the glatiramer acetate related drug substance, and the amino acid.

In one embodiment, the process comprises forming a liquid form of the glatiramer acetate related drug substance prior to forming the liquid form of the glatiramer acetate related drug substance and the amino acid. In another embodiment, the process comprises forming a liquid form of the amino acid prior to forming the liquid form of the glatiramer acetate related drug substance and the amino acid.

In one embodiment, the process comprises forming a liquid form of the laquinimod or pharmaceutically acceptable salt thereof prior to forming the liquid form of the laquinimod or pharmaceutically acceptable salt thereof and the amino acid. In another embodiment, the process comprises forming a liquid form of the amino acid prior to forming the liquid form of the laquinimod or pharmaceutically acceptable salt thereof and the amino acid.

In one embodiment, the liquid form is formed at stirring. In one embodiment, the process comprises passing the liquid form through a cellulose acetate syringe filter prior to forming a liquid form of the laquinimod or pharmaceutically acceptable salt thereof, the glatiramer acetate related drug substance, and the amino acid. In another embodiment, the process comprises storing the liquid form prior to forming a liquid form of the laquinimod or pharmaceutically acceptable salt thereof, the glatiramer acetate related drug substance, and the amino acid. In another embodiment, the liquid form is stored at 2-8′C. In one embodiment, the liquid form is clear.

The subject invention also provides a stable pharmaceutical composition comprising a therapeutically effective amount of laquinimod or pharmaceutically acceptable salt thereof, a therapeutically effective amount of glatiramer acetate related drug substance, and an amount of an amino acid, prepared by the processes described herein.

In one embodiment, the glatiramer acetate related drug substance is glatiramer acetate.

The subject invention also provides a sealed package comprising the compositions described herein.

In one embodiment, the sealed package after storage at 25° C. and at a relative humidity (RH) of 60% for at least 6 months is free of gelation. In one embodiment, the sealed package after storage at 25° C. and at a relative humidity (RH) of 60% for at least 12 months is free of gelation. In one embodiment, gelation is evaluated using the Ubbelohde tube method.

In one embodiment, the sealed package is a syringe. In one embodiment, the sealed package is a pre-filled syringe for administration. In another embodiment, the sealed package is a vial. In another embodiment, the sealed package is an ampule. In another embodiment, the sealed package is a cartridge. In another embodiment, the sealed package is an infusion.

The subject invention also provides a method for treating a subject afflicted with a form of autoimmune disease comprising administering to the subject a composition as described herein so as to thereby treat the subject.

The subject invention also provides a method for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome, comprising administering to the subject a composition as described herein so as to thereby treat and/or alleviate the symptom of multiple sclerosis in the subject.

The subject invention also provides a method for providing neuroprotection to a subject in need thereof comprising administering to the subject a composition as described herein so as to thereby provide neuroprotection to the subject.

The subject invention also provides use of a composition as described herein for treating a subject afflicted with an autoimmune disease.

The subject invention also provides use of a composition as described herein for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome.

The subject invention also provides use of a composition as described herein for providing neuroprotection to a subject in need thereof.

The subject invention also provides use of a composition as described herein in the manufacture of a medicament for treating a subject afflicted with an autoimmune disease.

The subject invention also provides use of a composition as described herein in the manufacture of a medicament for i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome.

The subject invention also provides use of a composition as described herein in the manufacture of a medicament for providing neuroprotection to a subject in need thereof.

The subject invention also provides a composition as described herein for use in a) treating a subject afflicted with an autoimmune disease, b) i) treating a subject afflicted with multiple sclerosis or presenting a clinically isolated syndrome, and/or ii) alleviating a symptom of multiple sclerosis in a subject afflicted with multiple sclerosis or presenting a clinically isolated syndrome, and/or c) providing neuroprotection to a subject in need thereof.

The subject invention also provides a pharmaceutical oral unit dosage form of a therapeutically effective amount of laquinimod or pharmaceutically acceptable salt thereof, a therapeutically effective amount of glatiramer acetate related drug substance, and an amount of an amino acid.

The subject invention also provides a package comprising: a) one or more unit doses, each such unit dose comprising i) a therapeutically effective amount of laquinimod or pharmaceutically acceptable salt thereof, ii) a therapeutically effective amount of glatiramer acetate related drug substance, and iii) an amount of an amino acid, and b) instructions for use of the one or more unit doses for i) treating a subject afflicted with an autoimmune disease, ii) treating a subject afflicted with multiple sclerosis or presenting a clinically isolated syndrome, and/or alleviating a symptom of multiple sclerosis in a subject afflicted with multiple sclerosis or presenting a clinically isolated syndrome, and/or iii) providing neuroprotection to a subject in need thereof.

For the foregoing embodiments, each embodiment disclosed herein is contemplated as being applicable to each of the other disclosed embodiments.

### Glatiramer Acetate

Glatiramer acetate mixtures, compositions, the process for the manufacture thereof, the use thereof for treatment of various conditions, and the corresponding dosages and regimens are described in, e.g., PCT International Application Publication Nos. WO 1998/30227, WO 2000/05250, WO 2000/18794, WO 2004/103297, WO 2006/029393, WO 2006/029411, WO 2006/083608, WO 2006/089164, WO 2006/116602. WO 2009/070298, WO 2011/022063, WO 2012/051106, WO 2003/048735, and WO 2011/008274, U.S. Patent Application Publication Nos. 2011-0230413 and 2008-027526, and U.S. Pat. Nos. 8,399,413, 8,008,258 and 7,556,767, each of which is hereby incorporated by reference in its entireties into this application.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in its entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar.

Capsule or tablets can be formulated and can be made easy to swallow or chew; other solid forms include granules and bulk powders. Tablets may contain suitable binders, lubricants, diluents, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrants include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and WO/2007/146248, each of which is hereby incorporated by reference into this application.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

A dosage unit may comprise a single compound or mixtures of compounds thereof. A dosage unit can be prepared for oral dosage forms, such as tablets, capsules, pills, powders, and granules.

### Terms

As used herein, and unless stated otherwise, each of the following terms shall have the definition set forth below.

As used herein, “laquinimod” means laquinimod acid or a pharmaceutically acceptable salt thereof, as well as derivatives as laquinimod such as deuterium enriched laquinimod, and salts thereof.

A “salt” is salt of the instant compounds which have been modified by making acid or base salts of the compounds. The term “pharmaceutically acceptable salt” in this respect, refers to the relatively non-toxic, inorganic and organic acid or base addition salts of compounds of the present invention. A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

As used herein, “about” in the context of a numerical value or range means ±10% of the numerical value or range recited or claimed.

An “amount” or “dose” of laquinimod as measured in milligrams refers to the milligrams of laquinimod acid present in a preparation, regardless of the form of the preparation.

The term “stable pharmaceutical composition” as used herein in connection with the composition according to the invention denotes a composition, which preserves the physical stability/integrity and/or chemical stability/integrity of the active pharmaceutical ingredient during storage. Furthermore, “stable pharmaceutical composition” is characterized by being free of gelation at 25° C./60% RH after at least 6 months.

As used herein, the term “glatiramer acetate related drug substance” (GARDS) is intended to include polypeptides with a predetermined sequence as well as mixtures of polypeptides assembled from the four amino acids glutamic acid (E), alanine (A), lysine (K), and tyrosine (Y); from any three of the amino acids Y, E, A and K, i.e. YAK, YEK, YEA or EAK; or from three of the amino acids Y, E, A and K and a fourth amino acid. Examples of glatiramer acetate related polypeptides are disclosed in U.S. Pat. Nos. 6,514,938 A1, 7,299,172 B2, 7,560,100 and 7,655,221 B2 and U.S. Patent Application Publication No. US 2009-0191173 A1, the disclosures of which are hereby incorporated by reference in their entireties. Glatiramer acetate related substances include glatiramoids.

As used herein, a “glatiramer acetate related drug substance or drug product” is a glatiramer acetate related drug substance or a glatiramer acetate related drug product.

As used herein a “glatiramoid” is a complex mixture of synthetic proteins and polypeptides of varying sizes assembled from four naturally occurring amino acids: L-glutamic acid, L-alanine, L-lysine, and L-tyrosine, in a defined molar ratio. Examples of glatiramoids include glatiramer acetate drug substance (e.g. Copaxone®) as well as glatiramoids other than Copaxone®, e.g. GA-Natco.

As used herein, a “clear” solution, formulation or form is a solution, formulation or form which appears clear or transparent by visible inspection. A turbid solution is not expected to appear clear to the eye.

As used herein, “treating” encompasses, e.g., inducing inhibition, regression, or stasis of a disease, disorder or condition, or ameliorating or alleviating a symptom of a disease, disorder or condition. “Ameliorating” or “alleviating” a condition or state as used herein shall mean to relieve or lessen the symptoms of that condition or state. “Inhibition” of disease progression or disease complication in a subject as used herein means preventing or reducing the disease progression and/or disease complication in the subject.

As used herein, “effective” as in an amount effective to achieve an end, i.e., “therapeutically effective amount”, means the quantity of a component that is sufficient to yield an indicated therapeutic response without undue adverse side effects (such as toxicity, irritation, or allergic response) commensurate with a reasonable benefit/risk ratio when used in the manner of this disclosure. For example, an amount effective to treat a subject afflicted with a form of multiple sclerosis. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Administering to the subject” means the giving of, dispensing of, or application of medicines, drugs, or remedies to a subject to relieve, cure, or reduce the symptoms associated with a condition, e.g., a pathological condition.

A “symptom” associated with multiple sclerosis includes any clinical or laboratory manifestation associated with multiple sclerosis and is not limited to what the subject can feel or observe.

As used herein, “a subject afflicted with multiple sclerosis” means a subject who was has been affirmatively clinically diagnosed to have multiple sclerosis which includes relapsing multiple sclerosis, relapsing-remitting multiple sclerosis, and Secondary Progressive multiple sclerosis.

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. “Pharmaceutically acceptable carrier” includes “fillers”, which fill out the size of a tablet or capsule, making it practical to produce and convenient for the consumer to use. By increasing the bulk volume, the fillers make it possible for the final product to have the proper volume for patient handling. “Pharmaceutically acceptable carrier” also includes “lubricants”, which prevent ingredients from clumping together and from sticking to the tablet punches or capsule filling machine. Lubricants also ensure that tablet formation and ejection can occur with low friction between the solid and die wall.

As used herein, a composition that is “free” of gelation means that the composition maintains its visual clarity and viscosity as measured using the Ubbelohde tube method, preferably after storage at 25° C. and at a relative humidity (RH) of 60% for at least 6 months.

It is understood that where a parameter range is provided, all integers within that range, and tenths and hundredth thereof, are also provided by the invention. For example, “0.15-0.35%” includes 0.15%, 0.16%, 0.17% etc. up to 0.35%.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### 1.53 mg/mL Laquinimod Sodium (Laq-Na) and 40 mg/mL Glatiramer Acetate (GA) with 40 mg/mL Mannitol

4.6 mg of laquinimod sodium (Laq-Na) was dissolved in 3.0425 g of and aqueous solution containing 40 mg/mL glatiramer acetate (GA) with 40 mg/mL mannitol as a tonicity agent.

Dissolution was achieved by effective stirring for 60 min at room temperature (RT) until a transparent solution containing 40 mg/mL GA and 1.53 mg/mL Laq-Na is formed (pH 5.89 at 22° C., osmolality 0.309 osmol/kg).

A gel was observed after 2 days.

### Example 2

### 4.63 mg/mL Lag-Na and 30 mg/mL GA

18.5 mg of Laq-Na was dissolved in 4 ml of purified water (PW) under effective stirring at RT for 150 min. 120 mg of GA was added at effective stirring to give a transparent solution containing 30 mg/mL GA and 4.63 mg/mL Laq-Na (pH 6.92).

A gel was observed.

### Example 3

### 3 mg/mL Lag-Na and 30 mg/mL GA with 9.0 mg/mL Sodium Chloride (NaCl)

123.4 mg of Laq-Na was dissolved in 41.121 g of PW at effective stirring with a magnet stirrer at PT. The solution looks transparent after 3 minutes (pH 7.45). 372.2 mg of sodium chloride (NaCl) was added as a tonicity agent (9 mg/mL) to ensure preparation of an isotonic or almost isotonic solution (0.290-0.300 osmol/kg). The solution was stirred for a few minutes at RT until the solution was transparent.

732.9 mg of GA was added to 24.4 g of the obtained solution containing 3 mg Laq-Na/mL and about 9 mg/mL NaCl. The solution was stirred at RT for one hour effectively but carefully, avoiding formation of air bubbles until the solution looked quite transparent (pH 5.9).

Following overnight storage at RT, a white gel was observed.

### Example 4

### 1 mg/mL Lag-Na and 30 mg/mL GA with about 8.1 mg/mL NaCl

60.1 mg of Laq-Na was dissolved in 60.001 g of PW under effective stirring at RT on a magnet stirrer. After 2 minutes the solution (Solution A) looked transparent.

44.5 mg of NaCl was added to 5.5055 g of Solution A as a tonicity agent and the solution was stirred for a few minutes at RT to get a transparent solution.

601.2 mg of GA DS (30 mg/mL GA) was added to 20.018 g (20 mL) of the obtained solution containing 1 mg/mL Laq-Na (osmolality 0.01-0.03 osmol/kg) and about 8.1 mg/mL NaCl. The solution was stirred at RT for one hour effectively but carefully, to avoid formation of air bubbles. The solution looked quite transparent, was not viscous and had an osmolality 0.274 osmol/kg.

After 2 days at RT, the solution became more turbid and more viscous, finally becoming a gel, a bit more transparent than in Example 5.

### Example 5A

### 1 mg/mL Lag-Na and 30 mg/mL GA

34.685 g of 0.05M potassium-phosphate buffer (pH 7.4) was added to 61 mg of Laq-Na. Concentration of Laq-Na was 1.017 mg/mL and osmolality was 0.113-0.114 osmol/kg.

602.5 mg of GA was added at stirring to 20 mL (20.019 g) of the transparent solution containing 1.017 mg/mL Laq-Na in 0.05 M buffer mixture (without NaCl). Transparent solution was formed (osmolality 0.1135—hypotonic).

Solution became almost transparent gels within a week at RT and at 2-8° C.

### Example 58

### 1 mg/mL Lag-Na and 30 mg/mL GA with 5.3 mg/mL NaCl

26.3183 g of 0.05M potassium-phosphate buffer was added to 61 mg of Laq-Na. Concentration of Laq-Na was 1.017 mg/mL and osmolality was 0.113-0.114 osmol/kg.

48 mg of NaCl (5.32 mg/mL NaCl) was added to 9 mL of the transparent solution containing 1.017 mg/mL Laq-Na in 0.05 M buffer mixture with stirring until a transparent solution was obtained.

233 mg of GA was added to 7.78 g of this isosmotic solution (osmolality 0.289 osmol/kg) with stirring at RT for 60 min, giving the transparent solution containing both 1 mg/mL Laq-Na and 30 mg/mL GA.

Solution became almost transparent gels within a week at RT and at 2-8° C.

### Example 6

### 3 mg/mL Lag-Na and 30 mg/mL GA with 5.0 mg/mL NaCl

73.11 mg of Laq-Na (3.0 mg/mL Laq-Na) was added to 24.35 g of sodium phosphate buffer (pH 7.1). 54.4 mg of NaCl (5 mg/mL NaCl) was added to 10.8 g of the solution. Osmolality of the resulting solution was 0.306 osmol/kg.

180.2 mg of GA (30 mg/mL GA) was added to 6 mL of the resulting solution (pH 7.10).

At 21′C, gel formed.

### Example 7

### 3 mg/mL Lag-Na and 30 mg/mL GA with 9.0 mg/mL NaCl

123.4 mg of Laq-Na (3 mg/mL Laq-Na) was added to PW at stirring at RT. Dissolution was achieved in 4 min. 372.3 mg of NaCl was added to the solution at stirring at RT. Dissolution was achieved in 3 min. 732 mg GA (about 30 mg/mL) was added to 24.4 g of the obtained solution at stirring at RT. Dissolution was achieved in 60 min.

Overnight, a white gel formed.

### Example 8

### 3 mg/mL Lag-Na and 10.7-40 mg/mL GA with 5.6 mg/mL NaCl

180.3 mg of Laq-Na was added to 60 mL of 0.05 potassium-phosphate buffer (pH 7.1) with gradual addition of GA from 10.7 mg/mL (slightly turbid solution) to 40 mg/mL (rather turbid solution).

The solution turned into gel very rapidly.

### Example 9

### 2.2 mg/mL Laq-Na and 5.0 mg/mL GA with 9.07 mg/mL NaCl

101.1 mg of GA (4.998 mg/mL GA) and 43.6 mg Laq-Na (2.1555 mg/mL Laq-Na) was added to 20.227 mL of PW. 183.4 mg of NaCl (9.067 mg/mL) was also added (pH 6.09).

A turbid solution was observed.

### Example 10

### 1 mg/mL Laquinimod Acid (Lag-Acid) and 30 mg/mL GA with 1.33 mg/mL L-Lysine and Meglumine

1.0203 g of meglumine solution was diluted by 3.5534 g of PW and then 8 mg of Laq-acid was added (Solution A).

1.341 g of the L-lysine stock solution containing 7.3 mg/mL in PW was added at stirring at RT to 3.6131 g of the GA stock solution containing 10 mg/mL in PW. To this clear solution, 2.2742 g of the preliminary prepared Solution A of Laq-acid in meglumine was added at stirring. The obtained sample contained 5 mg/mL GA, 1.35 mg/mL lysine and 1 mg/mL Laq-acid.

At first the obtained sample was not so turbid as having a GA concentration of 5 mg/mL. However a rapid growth of turbidity was observed, followed by gel-formation.

### Example 11

### 3 mg/mL Lag-Na and 40 mg/mL GA with PEG-400

402.5 mg of GA DS was added to 9.0048 g of 0.05M phosphate buffer (pH 7.1), 30.2 mg of Laq-Na, and 395.3 mg of PEG-400. Additional phosphate buffer was added to bring the total volume to 10 mL (616.8 mg).

In 3 days both solutions containing 3 mg/mL of Laq-Na and 40 mg/mL of GA (kept at RT and 2-8° C.) became viscous white gels.

### Example 12

### 2 mg/mL Lag-Na and 5 mg/mL GA

A transparent stock Solution A containing 10 mg/mL of GA-DS in PW was prepared by stirring 206.5 mg of GA DS in 20.644 g of PW at RT for 30 min (pH 5.90 at 22° C.). A transparent stock Solution B containing 4 mg/mL of Laq-Na in PW was prepared by stirring 166.1 mg of Laq-Na in 41.5173 g of PW for 10 min at PT (pH 8.86 at 22° C.).

5.1663 g of stock Solution A was mixed with 5.1575 g of stock Solution B. In 20 minutes of stirring at RT for a transparent solution (pH 6.08 at 21° C.) containing 2 mg/mL Laq-Na and 5 mg/mL of GA-DS, 59.1 mg of NaCl was added as a tonicity agent while stirring at RT.

The first crystals of NaCl added resulted in a slightly turbid solution; however, after 5 minutes of stirring it became more transparent (pH 6.01 at 22° C.).

After stirring overnight (for 16 h at RT) the solution looked transparent (pH 6.08 at 21° C.).

After storage at RT and 2-8° C. for week a solution started to become a gel: at first—a thin gel, however after 2 weeks it became a thick gel

### Example 13

### 0.7 mg/mL Lag-Acid and 30 m/mL GA

A saturated solution of laquinimod acid (3 mg/mL) was prepared by dissolving of 4.2283 g of Laq-acid in 0.1 M PBS (pH 7.4, 700 mL) for 3.5 h at RT. After decantation of the suspension the clear upper part was filtered through a paper (Whatman) filter. This clear, filtered solution contained about 1.5 mg/mL of laquinimod acid (Solution A).

60 mg/mL of glatiramer acetate in 0.1 M PBS (600 mL) was solubilized at RT for 3.5 h (Solution 8).

Equal volumes of Solutions A and B were mixed with stirring at RT and filtered through 0.2 μm polyethersulphone Parker filter 500 cm². The yield of the filtered solution containing 30 mg/mL GA and 0.7 mg/mL laquinimod was 420 mL.

The solution stored at RT and at 2-8° C. underwent slow gelation and growth of turbidity.

### Example 14

### 1.1 mg/mL Lag-Na and 32 mg/mL GA with 43.3 mg/mL Mannitol and 1.4 mg/mL Lysine

36 g of GA DS was weighed (on the basis of the dry powder, 4% of water in the DS batch was taken into account). Purified water was added to bring the total GA solution weight to 1029.4 g and stirring was performed for 30 min at RT until a nearly transparent, lump-free Solution A was obtained.

Mannitol powder (48 g) was added as a tonicity agent to Solution A with stirring at RT and afterwards stirring for 10 more minutes to obtain a lump-free Solution B.

Lysine-HCl (1.6249 g) was dissolved in 22.3596 g of purified water (pH 5.60) and added to the GA solution at stirring. The obtained solution after stirring for 10 more minutes at RT was clear and free from visible particles, and was filtered through a 0.2 μm cellulose acetate Sartorius syringe (with 17 cm² surface area) filter 1 (Solution C).

2.028 g of Laq-Na was dissolved in 68.8187 g of PW for 20 min until a clear solution of Laq-Na (pH 9.0) was obtained, which was then filtered through a similar 0.2 μm cellulose acetate Sartorius syringe filter 2 (Solution D).

The filtered Solution C (GA+mannitol+lysine+PW) (60 g) was sampled for investigational purposes and 60 g of the filtered Solution D (Laq-Na) were added dropwise with stirring to the remaining 993 g of Solution C. The resulting product was filtered through a similar 0.2 μm cellulose acetate Sartorius syringe filter 3. About 0.8 L was the final filtered product was obtained and manually filled into 1 cc USP type I glass pre-filled syringes (PFS) closed by brombutyl rubber stoppers, and into 10 cc USP type I glass clear vials (filling volume 5 cc) closed by brombutyl rubber stoppers.

Syringe functional testing was performed for the PFS samples and the gliding and breakout force was similar to the values for the PFS filled with the marketed drug Copaxone 20 mg/mL (breakout force 2 N, gliding force 1 N after 20 days of storage), which proved the absence of gelation.

Assay testing for both components corresponded to a concentration of 32.4 mg/mL glatiramer acetate and 1.1 mg/mL of laquinimod. Assay of both ingredients remained practically the same after storage at 5° C. and 25° C./60% RH, and molecular weight distribution values were similar to those for Copaxone marketed drug products. The DP solution in vials stored at 25° C. for 6 months and at 2-8° C. for 12 months and there were no essential changes in visual clarity and viscosity (measured using Ubbelohde tube method), which proved absence of gelation.

### Example 15

### Comparison of Formulations Using Various Amino Acids

15.2889 g of GA was weighed and dissolved with stirring for 30 min in 252.4 g of PW at RT prior to addition of 19.6 g of mannitol with stirring which went on for 10 minutes more followed by addition of 167.3 g of PW was added to wash out carefully the powder on the walls of the compounding vessel and stirring was completed after 5 more minutes. A clear solution essentially free of visible particles was obtained and contained 36.4 mg/mL GA and 46.7 mg/mL mannitol. The solution was filtered through a 0.2 μm cellulose acetate Sartorius syringe filter and stored at 2-8° C. before usage (pH 5.72) (Solution A).

221.9 mg of Laq-Na was weighed and dissolved in 12.6951 g of PW with stirring for 15 minutes at RT to give a clear solution containing 17.48 mg/mL of Laq-Na which was filtered through the similar filter and stored at 2-8° C. before usage (pH 9.05) (Solution B).

10.4 mg of amino acid proline was weighed and PW was added with stirring to bring the total weight to 246.1 mg. The stirring continued for 10 minutes at RT until complete dissolution of the amino acid. Then, 10.7722 g of Solution A was added at stirring prior to the addition by drops of 0.7 mL by pipette (however also weighed afterwards) of Solution 8.

Very similar amounts of Solutions A and B were added to the equimolar solutions of other amino acids (histidine, alanine, lysine, glycine). In case of a control no amino acid was used with the similar amounts of PW, and solutions A and B. The obtained samples containing GA (about 33.4 mg/mL), mannitol (about 42.8 mg/mL) and 1.07-1.09 mg/mL of Laq-Na were clear, except for the sample with histidine, which was almost clear.

### Example 16

### Comparison of Scaled-Up Formulations Using Various Amino Acids

For the scale-up of the formulations, in order to compare viscosity values at storage at 2-8° C. using Ubelohde tube, proportional quantities of each amino acid dissolved in PW were used, as well as of Solutions A and B to prepare not less than 30 g of the sample.

Usage of Ubbelohde tubes for evaluation of kinematic viscosity is based on measurements of time required to the tested liquid in the tube to pass the capillary inside from the upper mark to the lower mark. This old reproducible method has been mostly replaced by all kind of widespread Brookfield devices. However, stirring that occurs in Brookfield devices breaks the gel structure. In contrast, in an Ubbelohde tube, only slight partial disturbance of gel layers can take place. Therefore, we have found this method very useful for the systems inclined to gel-formation not only to evaluate the viscosity of the GA-Laq formulations, but also to observe the gelation process—in case of the presence of gel structure even where the product seems a liquid, decrease of the time results for each consecutive measurement proves the gel-formation.

Applying the Ubbelohde method, samples containing alanine (Sample A) and lysine (Sample B) stored in the Ubbelohde tubes at for 24 m at 2-8° C. was not accompanied by gel-formation, while a sample without amino acid (Sample C) showed slow gelation. Samples with glycine (Samples D) and proline (Sample E) differed not so much from the control—Sample C (in the amino acid concentrations used), while the histidine sample (Sample F) showed accelerated gel-formation in comparison to Sample C.

### Example 17

10.21 g of GA was weighed and dissolved at stirring for 60 min in 280.28 g of Water for Injection at RT prior to addition of 13.1 g of mannitol at stirring which went on for 20 minutes more. A clear solution essentially free of visible particles was obtained containing 35.14 mg/mL GA and 43.15 mg/mL mannitol. The solution was filtered through a 0.2 μm cellulose acetate Sartorius syringe filter and stored at 2-8° C. before usage (pH 5.75)(Solution A).

224 g of Laq-Na was weighed and dissolved in 12.815 g of Water for Injection at stirring for 15 minutes at RT to give a clear solution containing 17.48 mg/mL of Laq-Na which was filtered through the similar filter and stored at 2-8° C. before usage (pH 9.06) (Solution B).

33 mg of histidine was solubilized in 275 mg of Water for Injection after shaking for a minute at RT and 10.868 g of a clear Solution A was added to a rather turbid aqueous suspension of the histidine. The clear solution was obtained after stirring at RT.

0.716 mg of clear Solution B was added drop by drop at stirring at RT resulting into a clear solution containing 34 mg/mL of GA, 42 mg/mL of mannitol, 1.11 mg/mL of Laq-Na and 2.93 mg/mL of histidine (Solution C). After stirring overnight at RT the solution remained clear and its samples remained clear both at RT and 5° C.

To 6.015 g of Solution C additional amounts of Laq-Na were added drop by drop at stirring at RT. The solutions obtained were clear at 2-8° C. and at RT at Laq-Na concentrations 1.28, 1.49, 1.75 mg/mL. The addition of Laq-Na up to 2.04 mg/mL resulted in a clear solution containing 32.1 mg/mL GA, 39.6 mg/mL Mannitol and 2.75 mg/mL histidine, which after stirring overnight at RT became a bit turbid (and remained as such afterwards) and thixotropic: a liquid at shaking or stirring which without shaking or stirring became a weak gel, and such transitions could occur for many days of observation.

Histidine concentration lower than 1.33 mg/mL could not solubilize additional portions of Laq-Na—1.23, 1.39, 1/54 mg/mL—all of them gave thixotropic products with increased turbidity and increased ability to gel-formation.

Similar results (higher Laq-Na solubilization effect (higher Laq-Na dose incorporated in a clear product) at increase of amino acid concentration) was observed for other amino acids:

With increase of glycine from 0.6 mg/mL to 4.2 mg/mL, it was possible to incorporate 1.51 mg/mL of Laq-Na instead of 1.05-1.07 mg/mL.

With increase of proline from 0.9 mg/mL to 2.45 mg/mL, it was possible to incorporate 1.55 mg/mL of Laq-Na instead of 1.06-1.09 mg/mL.

With increase of lysine from 1.39 mg/mL to 3.38 mg/mL, it was possible to incorporate 1.88 mg/mL of Laq-Na instead of 1.06-1.09 mg/mL.

Table 1 below summarizes that experiments on fixed-dose formations of laquinimod and glatiramer acetate.

TABLE 1

| | Laq | GA | | | | | |
|---|---|---|---|---|---|---|---|
| | Conc | Conc. | | Tonicity | Additional | | |
| N | mg/mL | mg/mL | Solvent | agent | ingredients | Result | Note |
| 1 | 1.53 | 40 | PW | Mannitol | — | gelation | Isosmotic |
| | | | | 40 mg/mL | | | (0.310) |
| 2 | 4.63 | 30 | PW | — | — | gel | Very |
| | | | | | | | hypotonic |
| 3 | 3.1 | 40 | 0.05M | — | — | gel | rather |
| | | | K- | | | | hypotonic |
| | | | Phosphate | | | | (0.121) |
| | | | buffer pH | | | | |
| | | | 7.2 | | | | |
| 4 | 1 | 30 | PW | NaCl | — | Less turbid | Nearly |
| | | | | 8.1 mg/mL | | than ##4-6, | isotonic |
| | | | | | | slow | (0.280) |
| | | | | | | gelation | |
| 5 | 1 | 30 | 0.05M | — | — | Growth of | rather |
| | | | K- | | | turbidity, | hypotonic |
| | | | Phosphate | | | then slow | (0.114) |
| | | | buffer pH | | | gelation | |
| | | | 7.2 | | | | |
| 6 | 1 | 30 | “—” | NaCl | | Growth of | Isotonic |
| | | | | 5.3 mg/mL | | turbidity, | (0.290) |
| | | | | | | then slow | |
| | | | | | | gelation | |
| 7 | 3 | 30 | 0.05M | NaCl | — | Turbid, | isotonic |
| | | | Na | 5.0 mg/mL | | rapid | (0.306) |
| | | | Phosphate | | | gelation | |
| | | | buffer pH | | | | |
| | | | 7.1 | | | | |
| 8 | 3 | 30 | PW | NaCl | — | Growth of | A bit |
| | | | | 9.0 mg/mL | | turbidity, | hypertonic |
| | | | | | | rapid | (0.327) |
| | | | | | | gelation | |
| 9 | 3 | 10.7-40 | 0.05M | NaCl | — | Growth of | addition of |
| | | | Na | 5.6 mg/mL | | turbidity for | GA to Laq— |
| | | | Phosphate | | | more GA, | Na solution |
| | | | buffer pH | | | rapid | by steps |
| | | | 7.1 | | | gelation | |
| 10 | 1.2- | 40 | “—” | NaCl | — | Growth of | Addition of |
| | 2.95 | | | 5.9 mg/mL | | turbidity | Laq—Na to |
| | | | | | | and gelation | GA solution |
| | | | | | | for more | |
| | | | | | | Laq—Na | |
| 11 | 1.4- | 40 | PW | NaCl | — | Better than | “—” |
| | 2.93 | | | 8.9 mg/mL | | #10, but | |
| | | | | | | also growth | |
| | | | | | | of turbidity | |
| | | | | | | and gelation | |
| | | | | | | for more | |
| | | | | | | Laq—Na | |
| 12 | 3 | 40 | 0.07M | — | — | gel | Another |
| | | | Na- | | | | buffer |
| | | | Acetate | | | | |
| | | | buffer pH | | | | |
| | | | 6.15 | | | | |
| 13 | 3 | 40 | “—” | — | PEG-400 | Viscous gel | PEG-400 |
| 14 | 3 | 40 | 0.05M | — | PEG-400 | Viscous gel | PEG-400 |
| | | | Na | | | | |
| | | | Phosphate | | | | |
| | | | buffer pH | | | | |
| | | | 7.1 | | | | |
| 15 | 3 | 40 | “—” | NaCl | — | Turbid | GA and |
| | | | | 5.1 mg/mL | | solution, | Laq—Na |
| | | | | | | rapid | added from |
| | | | | | | gelation | stock |
| | | | | | | | solutions |
| | | | | | | | (60 mg/mL |
| | | | | | | | and |
| | | | | | | | 9 mg/mL) |
| 16 | 3 | 40 | PW | NaCl | — | A bit less | |
| | | | | 8.9 mg/mL | | turbid | |
| | | | | | | solution, | |
| | | | | | | gelation | |
| 17 | 2.2 | 5.0 | PW | NaCl | — | Turbid | Laq—Na |
| | | | | 9.07 mg/mL | | solution, | added to |
| | | | | | | gelation, | GA |
| | | | | | | better, than | solution, |
| | | | | | | ##7-9, 12-16 | less GA |
| 18 | 2.0 | 5.0 | 0.05M | NaCl | — | More turbid | “—” |
| | | | Na | 5.2 mg/mL | | Solution | |
| | | | Phosphate | | | than #17, | |
| | | | buffer pH | | | gelation | |
| | | | 7.1 | | | | |
| 19 | 2.0 | 5.0 | PW | — | — | After | Laq—Na and |
| | | | | | | stirring | GA added |
| | | | | | | overnight- | from stock |
| | | | | | | transparent | solutions. |
| | | | | | | solution, in | Prolonged |
| | | | | | | a week- | stirring |
| | | | | | | gelation | |
| 20 | 2.0 | 5.0 | Fresh | NaCl | — | Turbid, | “—” |
| | | | 0.05M | 5.7 mg/mL | | gelation | |
| | | | Na | | | | |
| | | | Phosphate | | | | |
| | | | buffer pH | | | | |
| | | | 7.1 | | | | |
| 21 | 2.0 | 5.0 | PW | — | Meglumin | gelation | Meglumin |
| | | | | | 3.36 mg/mL | | solution |
| | | | | | | | added to |
| | | | | | | | both |
| | | | | | | | solutions |
| | | | | | | | prepared |
| | | | | | | | from stock |
| | | | | | | | solutions |
| 22 | 0.9 | 5.0 | PW | — | Meglumin | Turbid, | Laq-acid |
| | Laq- | | | | 8.5 mg/mL | gelation | instead of |
| | acid | | | | | | Laq—Na |
| 23 | 1.28 | 5.0 | PW | — | 1.33 mg/mL | Clear | All 3 |
| | | | | | L-Lysine | solution, no | components |
| | | | | | | gelation | added as |
| | | | | | | | solutions in |
| | | | | | | | PW |
| 24 | 1 | 30 | PW | — | 1.33 mg/mL | Not | GA+ + Lys + |
| | Laq- | | | | L-Lysine + | transparent, | Laq-acid |
| | acid | | | | Meglumin | gel-like | instead of |
| | | | | | | | Laq—Na |
| 25 | 1.24 | 30 | PW | — | 1.39 mg/mL | Getting | “—” |
| | Laq- | | | | L-Lysine + | more turbid | |
| | acid | | | | Meglumin | on storage | |
| 26 | 1.26 | 30 | PW | — | 1.35 mg/mL | Clear | Similar to |
| | | | | | L-Lysine | solution, no | #23, The |
| | | | | | | gelation | best |
| | | | | | | | formulation. |
| 27 | 1.9 | 30 | 0.05M | — | 1.4 mg/mL | At RT clear | Lys and |
| | | | Histidine | | L-Lysine | slow gelation, | Laq—Na in |
| | | | buffer | | | at 5° a weak | Histidine |
| | | | (HB) (GA | | | gel becomes | buffer |
| | | | in PW) | | | solution, | |
| | | | | | | better than | |
| | | | | | | #29 | |
| 28 | 1.9 | 30 | “—” for | — | 1.4 mg/mL | pH 7.0. | Laq—Na |
| | | | Laq—Na; | | L-Lysine | Slow | (9 mg/mL |
| | | | PW for | | | gelation, | stock |
| | | | Lysine & | | | better than | solution); |
| | | | GA | | | #27 and #29 | |
| 29 | 1.9 | 30 | Only 0.05M | — | 1.4 mg/mL | gelation at | |
| | | | HB | | L-Lysine | storage, pH | |
| | | | | | | 7.34 | |
| 30 | 2.8 | 30 | PW | — | 1.4 mg/mL | A bit turbid, | More Laq—Na |
| | | | | | L-Lysine | very | |
| | | | | | | viscous, gel- | |
| | | | | | | like at RT, | |
| | | | | | | pH 6.1, | |
| | | | | | | worse than | |
| | | | | | | ##23, 26-28 | |
| 31 | 2.0 | 30 | PW | — | 2 mg/mL | Almost clear, | Less Laq—Na |
| | | | | | L-Lys | pH 6.11, | than in 30 |
| | | | | | | slow gelation | |
| | | | | | | at storage, | |
| | | | | | | better than | |
| | | | | | | ##30, 33, | |
| 32 | 1.62 | 30 | PW | — | 2.34 mg/mL | Almost | Less Laq—Na |
| | | | | | L-Lysine | clear, pH | than in 31 |
| | | | | | | 6.16, better | |
| | | | | | | than #31 | |
| 33 | 2.2 | 30 | PW | — | 2.34 mg/mL | Almost | More Laq—Na |
| | | | (GA, Lys) | | L-Lysine | clear, a bit | than in # |
| | | | HB—Laq | | | viscous, pH | 31 |
| | | | Na, | | | 7.03, better | |
| | | | 9.66 mg/mL | | | than #30, | |
| | | | | | | worse than | |
| | | | | | | ## 31-32 | |
| 34 | 1.4 | 30 | GA in | — | 2.5 mg/mL | Nice, but at | HB while |
| | | | 0.05M HB, | | L-Lysine | storage a bit | #26-in |
| | | | Laq—Na | | | more turbid | PW, and |
| | | | 9 mg/mL | | | than #26, | more Lys |
| | | | & Lys-PW | | | Best among | |
| | | | | | | ## 30-34 | |
| 35 | 1.265 | 30 | PW | — | 1.33 mg/mL | Clear, no | A small |
| | | | (Laq—Na | | L-Lysine | gelation | scale up of |
| | | | 4 mg/mL) | | | | #26 |
| | | | | | | | (reproducibility) |
| 36 | 1.258 | 30 | PW | — | 1.36 mg/mL | Clear, in | As # 35 but |
| | | | (GA, Lys) | | L-Lysine | 1.5 m a bit | Laq—Na in |
| | | | HB— | | | turbid | HB, as in |
| | | | Laq—Na | | | | #35 less |
| | | | 4 mg/mL | | | | concentrated |
| | | | | | | | Laq—Na |
| 37 | 2.0 | 40 | PW | — | 1.5 mg/mL | Almost | More, Laq—Na |
| | | | (GA, Lys) | | L-Lysine | clear, after | & L-Lys |
| | | | HB— | | | 1 m 5° C. | than in #36 |
| | | | Laq—Na | | | turbid | |
| | | | 16 mg/mL | | | | |
| 38 | 2.14 | 40 | “—” | — | “—” | Almost | A bit more |
| | | | | | | clear, after | Laq—Na than |
| | | | | | | 3 m 5° C. | in #37 |
| | | | | | | more turbid | |
| | | | | | | than #37 | |
| 39 | 1.1 | 40 | PW—GA | — | 2 mg/mL | Clear, in 1 m | More Lys, |
| | | | HB-Lys | | L-Lysine | 5° C. turbid | less Laq |
| | | | & Laq—Na | | | | than in |
| | | | | | | | ##37-38 |
| 40 | 1.77 | 35 | PW | — | 2.23 mg/mL | Clear, in 1 m | More Lys & |
| | | | (GA, Lys) | | L-Lysine | 5° C. turbid | Laq |
| | | | HB— | | | | than in #39 |
| | | | Laq—Na | | | | |
| 41 | 0.97 | 35 | PW | — | 1.66 mg/mL | Clear in | More GA & |
| | | | HB— | | L-Lysine | 3.5 m, then a | a bit less |
| | | | Laq—Na | | | bit turbid | Laq than in |
| | | | | | | | #26, mo, |
| 42 | 0.99 | 35 | PW | — | 2.6 mg/mL | Clear after | Very |
| | | | (GA, Lys) | | L-Lysine | 3 m, | hypotonic |
| | | | HB— | | | osmolality | without a |
| | | | Laq—Na | | | 0.044 | tonicity |
| | | | | | | | agent |
| 43 | 1.23 | 30 | PW | Mannitol | 1.32 mg/mL | Too low | Still |
| | | | | 22.3 mg/mL | L-Lysine | Mannitol | hypotonic |
| | | | | | | osmolality | solution |
| | | | | | | 0.178 | |
| 44 | 1.26 | 30 | PW | 5.9 mg/mL | 1.35 mg/mL | Clear, in | NaCl as a |
| | | | | NaCl | L-Lysine | 1 m a bit | tonicity |
| | | | | | | turbid, | agent, a bit |
| | | | | | | Osmolality | hypertonic |
| | | | | | | 0.234 | |
| 45 | 1.244 | 30 | PW | Mannitol | 1.39 mg/mL | Clear, stable | Still a bit |
| | | | | powder | L-Lysine | Osmolality | hypotonic |
| | | | | 35.6 mg/mL | | 0.247 | |
| 46 | 1.2 | 30 | PW (Laq— | Mannitol | 1.47 mg/mL | Clear, slow | Finally 0.04M |
| | | | Na in 0.1M | powder | L-Lysine | growth of | NaHCO₃, |
| | | | NaHCO₃) | 36 mg/mL | | turbidity | nearly |
| | | | | | | and | isotonic (as |
| | | | | | | viscosity, | Cop 20)/a |
| | | | | | | Osmolality | bit hypo |
| | | | | | | 0.275 | |
| 47 | 1.26 | 30 | PW (Laq— | Mannitol | 1.35 mg/mL | Clear, slow | Finally 0.04M |
| | | | Na in 0.1M | (solution) | L-Lysine | growth of | NaHCO₃, |
| | | | NaHCO₃) | 35.9 mg/mL | | turbidity & | Still a bit |
| | | | | | | viscosity, | hypotonic |
| | | | | | | Osmolality | |
| | | | | | | 0.264 | |
| 48 | 1.0 | 30 | PW (Laq— | Mannitol | 1.9 mg/mL | Clear, | More Lysine |
| | | | Na in 0.1M | sol. | L-Lysine | turbidity | Finally 0.04M |
| | | | NaHCO₃) | 35.9 mg/mL | | growth in | NaHCO₃, |
| | | | | | | 3 m | |
| 49 | 1.0 | 30 | PW | Mannitol | 1.8 mg/mL | Clear for 6 m | Filtered |
| | | | | sol. | L-Lysine | storage, a bit | Laq—Na |
| | | | | 36.6 mg/mL | | turbid after | |
| | | | | | | 7.5 m | |
| 50 | 1.0 | 30 | PW | Mannitol | 1.0 mg/mL | A bit turbid | Less Lys |
| | | | | sol. | L-Lysine | alter 6 m | |
| | | | | 36.6 mg/mL | | than #51 | |
| 51 | 1.0 | 29.8 | PW | Mannitol | 1.8 mg/mL | 6 m clear | More Lysine |
| | | | | sol. | L-Lysine | | |
| | | | | 36.4 mg/mL | | | |
| 52 | 1.0 | 29.9 | PW | Mannitol | 1.37 Lysine | 6 m clear | Reproduction |
| | | | (Laq—Na | sol | | | of # 26 |
| | | | 4 mg/mL | S | | | |
| | | | | 36.5 mg/mL | | | |
| 53 | 1.1 | 32 | PW | 43.3 mg/mL | 1.4 mg/mL | Clear, stable, | Filtered |
| | | | | Mannitol | Lysine | no gelation | scale-up |
| | | | | | | nice | |
| | | | | | | filterability + | |
| | | | | | | See example | |
| 54 | 1.04 | 29.9 | PW | 36.5 mg/mL | 0.8 mg/mL | 6 m clear, | Less Lys |
| | | | | Mannitol | Lysine | after 6.5 m a | |
| | | | | | | bit turbid | |
| 55 | 1.01 | 30 | PW | 36.6 mg/mL | 0.8 mg/mL | Worse than | Less Lysine |
| | | | | Mannitol | Lysine | # 54, 5 m | |
| | | | | | | clear, then | |
| | | | | | | slow | |
| | | | | | | turbidity | |
| | | | | | | growth and | |
| | | | | | | gelation | |
| 56 | 1.13 | 6 | PW | 7.3 mg/mL | 0.55 Lysine | Clear, no | Low GA, |
| | | | | Mannitol | | gelation at | Low Lys, |
| | | | | | | storage | very |
| | | | | | | | hypotonic! |
| 57 | 0.97 | 32 | PW | Mannitol | 1.53 mg/mL | clear | For |
| | | | | 41 mg/mL | Lysine | | viscosity |
| | | | | | | | monitoring |
| 58 | 1.04 | 29 | PW | Mannitol | 1.53 mg/mL | clear | reproducibility |
| | | | | 40 mg/mL | Lysine | | |
| 59 | 1.04 | 29 | PW | Mannitol | 0.85 mg/mL | Clear, a bit | Less Lysine, |
| | | | | 37.2 mg/mL | Lysine | Worse than | lower GA |
| | | | | | | # 57-58 at | conc., |
| | | | | | | storage | higher Laq—Na |
| | | | | | | | conc. |
| 60 | 1.06 | 29 | PW | Mannitol | 3.38 mg/mL | Clear, | More Lys, |
| | | | | 37.2 mg/mL | Lysine | Worse than | less PW |
| | | | | | | # 54, | |
| 61 | 1.05 | 29 | PW | Mannitol | 1.58 mg/mL | clear | |
| | | | | 37.2 mg/mL | Lysine | | |
| 62 | 1.1 | 5.0 | PW | Mannitol | 2.78 mg/mL | clear | Lower GA, |
| | | | | 37.6 mg/mL | Lysine | | higher Lys |
| 63 | 2.0 | 4.7 | PW | Mannitol | 4.20 mg/mL | Almost | Higher Laq, |
| | | | | 36.7 mg/mL | Lysine | clear, gets | lower GA, |
| | | | | | | turbid | highest Lys |
| 64 | 1.6 | 5.0 | PW | Mannitol | 1.8 mg/mL | A bit turbid, | |
| | | | | 38.2 mg/mL | L-Lysine | slow growth | |
| | | | | | | of turbidity | |
| 65 | 2.76 | 30 | PW | Mannitol | 1.33 mg/mL | Very turbid | High Laq |
| | | | | 40 mg/mL | L-Lysine | | cone |
| 66 | 2.15 | 30 | PW | Mannitol | 1.34 mg/mL | turbid | Another |
| | | | | 40 mg/mL | L-Lysine | | variant of |
| | | | | | | | adding |
| | | | | | | | excipients |
| 67 | 2.42 | 34 | PW | Mannitol | 2.12 mg/mL | Very turbid | Tween |
| | | | | 41 mg/mL | Lysine | | 80 + Lys to |
| | | | | | 2.5 mg | | dissolve |
| | | | | | Tween 80 | | more Laq—Na |
| 68 | 1.0 | 32 | PW | 46.6 mg/mL | 1.4 mg/mL | Clear, stable | More scale- |
| | | | | Mannitol | L-Lysine | | up for |
| | | | | | | | filtration |
| | | | | | | | studies |
| 69 | 0.7 | 30 | PBS | — | — | Clear, at | Laq-acid |
| | (Laq- | | | | | storage- | and GA in |
| | acid) | | | | | turbid | PBS |
| 70 | 0.74 | 30 | PW | 36.5 mg/mL | (Without | Almost | Laq—Na by |
| | | | | Mannitol | AK) | Clear, | steps for |
| | | | | | | slowly | comparison |
| | | | | | | getting more | with |
| | | | | | | viscous | samples |
| | | | | | | | containing |
| | | | | | | | various |
| | | | | | | | amino acids |
| 71 | 0.74 | 30 | PW | 36.5 mg/mL | 1.1 mg/mL | Clear, pH | |
| | | | | Mannitol | Lysine | 5.93 | |
| 72 | 1.07 | 33.2 | PW | 42.6 mg/mL | 0.6 mg/mL | Clear, pH | |
| | | | | Mannitol | Glycine | 5.8 | |
| 73 | 1.09 | 33.4 | PW | 42.8 mg/mL | 0.9 mg/mL | Clear | |
| | | | | Mannitol | Proline | | |
| 74 | 1.08 | 33.4 | PW | 42.8 mg/mL | 0.8 mg/mL | pH 5.72 | |
| | | | | Mannitol | Alanine | | |
| 75 | 1.08 | 33.4 | PW | 42.8 mg/mL | 1.2 mg/mL | Almost | |
| | | | | Mannitol | Histidine | clear | |
| 76 | 1.09 | 33.4 | PW | 42.8 mg/mL | 1.39 mg/mL | Clear | |
| | | | | Mannitol | Lysine | | |
| 77 | 1.08 | 33.4 | PW | 42.8 mg/mL | (Without | Clear | “—” (control) |
| | | | | Mannitol | AK) | | |
| 78 | 1.05 | 33.4 | PW | 43 mg/mL | 0.6 mg/mL | Clear, Slow | Scale-up for |
| | | | | Mannitol | Glycine | gelation, | viscosity |
| | | | | | | similar to | and other |
| | | | | | | the control | testing |
| 79 | 1.06 | 33.4 | PW | 43 mg/mL | 0.71 mg/mL | Stable, clear | “—” |
| | | | | Mannitol | Alanine | no gelation | |
| | | | | | | similar to | |
| | | | | | | Lys | |
| 80 | 1.06 | 33.4 | PW | 43 mg/mL | 0.91 mg/mL | Clear, slow | “—” |
| | | | | Mannitol | Proline | gelation | |
| | | | | | | similar to | |
| | | | | | | the control | |
| 81 | 1.06 | 33.4 | PW | 43 mg/mL | 1.2 mg/mL | Worst | “—” |
| | | | | Mannitol | Histidine | among all | |
| | | | | | | amino acids | |
| | | | | | | tested: | |
| | | | | | | accelerating | |
| | | | | | | gelation | |
| | | | | | | even in | |
| | | | | | | comparison | |
| | | | | | | to control | |
| | | | | | | #83 | |
| 82 | 1.06 | 33.4 | PW | 43 mg/mL | 1.39 mg/mL | Stable, | “—” |
| | | | | Mannitol | Lysine | clear, no | |
| | | | | | | gelation | |
| 83 | 1.075 | 33.4 | PW | 43 mg/mL | (Without | Almost | “—” (control) |
| | | | | Mannitol | AK) | clear, slow | |
| | | | | | | gelation | |
| 84 | 1.11 | 34.0 | PW | 42 mg/mL | 2.93 mg/mL | Clear | More than |
| | 1.31 | 33.6 | | Mannitol | Histidine | solution at | double His |
| | 1.53 | 33.1 | | 41.5 mg/mL | 2.89 | RT & 5° C. | than in |
| | 1.79 | 32.6 | | M | 2.86 | Clear | ##75, 81 |
| | 2.04 | 32.1 | | 40.9 | 2.81 | solution at | |
| | | | | 40.3 | 2.76 | 5° C. | |
| | | | | 39.6 | | “—” | |
| | | | | | | “—” | |
| | | | | | | Thixotropic | |
| | | | | | | & a bit | |
| | | | | | | turbid at RT | |
| 85 | 1.09 | 34 .0 | PW | 42 mg/mL | 1.33 mg/mL | Clear | 10% more |
| | | | | Mannitol | Histidine | solution at | His than 75, |
| | | | | | | RT & 5° C. | 81 |
| | | | | | | | Further |
| | | | | | | | addition of |
| | | | | | | | Laq—Na |
| | | | | | | | (1.23, 1.39, |
| | | | | | | | 1.54 mg/mL) |
| | | | | | | | gave a bit |
| | | | | | | | turbid, then |
| | | | | | | | more turbid |
| | | | | | | | thixotropic |
| | | | | | | | products (R- |
| | | | | | | | 12602) |
| 86 | 1.05 | 30.7 | PW | 37.7 mg/mL | 4.21 mg/mL | Clear | 4-times |
| | 1.45 | 30.0 | | Mannitol | Histidine | solution at | more His |
| | | | | 36.8 | 4.11 | RT & 5° C. | than in |
| | | | | | | | ##75, 81 |
| 87 | 1.13 | 30.7 | PW | 37.6 mg/mL | 2.51 mg/mL | Clear | More than |
| | 1.55 | 29.9 | | Mannitol | Proline | solution at | double Pro |
| | | | | 36.6 | 2.45 | RT & 5° C. | than in ##73, |
| | | | | | | Clear | 80 |
| | | | | | | solution at | |
| | | | | | | 5° C. | |
| 88 | 1.43 | 29.7 | PW | 36.7 mg/mL | 3.48 mg/mL | Clear | More than |
| | 1.88 | 28.9 | | Mannitol | Lysine | solution at | double Lys |
| | | | | 35.7 | 3.38 | RT & 5° C. | than in |
| | | | | | | Clear | ##53, 68, 76 |
| | | | | | | solution at | |
| | | | | | | RT & 5° C. | |
| 89 | 1.05 | 31.3 | PW | 38.4 mg/mL | 4.32 mg/mL | Clear | More than |
| | 1.51 | 30.4 | | Mannitol | Glycine | solution at | double |
| | | | | 37.3 | 4.20 | RT & 5° C. | Glycine |
| | | | | | | “—” | than in |
| | | | | | | | ##53, 72, 78 |
| 90 | 1.1 | 31.3 | PW | — | 3.3 mg/mL | Slow | Without |
| | | | | | Histidine | gelation only | Mannitol |
| | | | | | | at RT (not at | hypotonic |
| | | | | | | 5° C.) | |
| 91 | 1.1 | 30.0 | PW | — | 4.4 mg/mL | Practically no | Without |
| | | | | | Glycine | gelation even | Mannitol |
| | | | | | | at RT | hypotonic |
| 92 | 1.4 | 29.7 | PW | — | 3.4 mg/mL | A weak | Without |
| | | | | | Lysine | thyxotropic | Mannitol |
| | | | | | | gel only at | hypotonic |
| | | | | | | RT | |
| 93 | 1.2 | 30.0 | PW | — | 2.9 mg/mL | A weak | Without |
| | | | | | Proline | thyxotropic | Mannitol |
| | | | | | | gel only at | hypotonic |
| | | | | | | RT | |
| 94 | — | 32.0 | PW | 35.0 | 3.2 mg/mL | No gelation | Almost |
| | | | | | Histidine | even at RT | isotonic |
| | | | | | | | solution |
| 95 | — | 31.1 | PW | 38.2 | 4.6 mg/mL | No gelation | Isotonic |
| | | | | | Glycine | even at RT | solution |
| 96 | — | 31.3 | PW | 38.4 | 3.8 mg/mL | No gelation | Isotonic |
| | | | | | Lysine | even at RT | solution |
| 97 | — | 30.6 | PW | 35.1 | 2.9 mg/mL | No gelation | Almost |
| | | | | | Proline | even at RT | Isotonic |
| | | | | | | | solution |
| 98 | 1.2 | — | PW | 39.0 | 3.2 mg/mL | No gelation | Almost |
| | | | | | Histidine | even at RT | Isotonic |
| | | | | | | | solution |
| 99 | 1.1 | — | PW | 38.5 | 4.6 mg/mL | No gelation | Almost |
| | | | | | Glycine | even at RT | Isotonic |
| | | | | | | | solution |
| 100 | 1.1 | 29.4 | PW | — | 21.7 mg/mL | After 6 m RT | More |
| | | | | | Glycine | weak | Glycine. |
| | | | | | | thixotropic | Hypotonic |
| | | | | | | gel | solution |
| 101 | 1.1 | — | PW | 37.5 | 2.9 mg/mL | No gelation | Almost |
| | | | | | Proline | even at RT | Isotonic |
| | | | | | | | solution |
| 102 | 1.5 | — | PW | 37.4 | 3.7 mg/mL | No gelation | Almost |
| | | | | | Lysine | even at RT | Isotonic |
| | | | | | | | solution |
| 103 | 1.2 | 29.6 | PW | — | 13.2 mg/mL | After 3 m RT | More |
| | | | | | Proline | weak | Proline. |
| | | | | | | thixotropic | Hypotonic |
| | | | | | | gel | solution |

### Methods:

For the gelation process study, several modern sophisticated methods were used: ultracentrifugation for fractionation of GA-LAQ mixture prior to chromatographic methods such as HPLC-SEC, LC-MS; cryo-transmission electron microscopy (Cryo-TEM); circular dichroism; amino acid analysis.

For selection of better formulations, viscosity testing using a Ubbelohhde viscometer (tube size 1) was performed.

Osmolality was evaluated using Osmomat 030-0.

In addition, for the characterization of the best scaled-up formulation comprising GA LAQ (1.1 mg/mL), L-lysine (1.4 mg/mL) and mannitol (43 mg/mL) as a tonicity agent, HPLC-SEC (size-exclusion chromatography) was used to evaluate the GA assay and molecular-weight distribution, while HPLC was used for LAQ assay. Clarity was evaluated using a 2100 AN turbidimeter. Functional tests for the drug product filled in pre-filled syringes were performed to evaluate Breakout and Gliding forces.

### DISCUSSION

Developing formulations containing laquinimod and GA is particularly difficult because laquinimod and GA have different pH optimums in terms of stability and solubility and because of undesired effects, mainly gelation or the formation of turbid formulations in the presence of various buffers, solvents and surface active agents.

GA requires a pH not exceeding 7.0, preferably 5.5-6.2, to limit degradation. Laquinimod, on the other hand, is more soluble and stable in alkaline media. In addition, the presence of laquinimod even at low concentrations (0.7-2.0 mg/mL) with GA has been found to cause gelation of GA and/or gelation of the final formulations.

Experiments show that fixed-dose GA-laquinimod formulations containing 5-40 mg/mL of GA and 0.7-5 mg/mL of laquinimod were not transparent liquid forms, and more or less rapidly turned into gels. The higher the concentration of GA or laquinimod, the more significant the growth of turbidity, the greater the viscosity, and the faster the rate of gelation was observed. The method and order of mixing of components were also important factors.

Numerous studies of various buffers, solvents and surface-active agents with various forms of laquinimod (acid and salts) have consistently led to viscous solutions that gradually form gels, or lead to sedimentation or to rapid formation of turbid formulations.

These studies show that use of various buffers was not preferable to the use of purified water. The use of various buffers caused turbidity of GA, or turbidity and/or gelation of the final formulations. In particular, phosphate salt buffers (sodium, potassium, and sodium-potassium salts), acetate, and hydrocarbonate all providing pH of 5.5-8 in numerous experiments, were found to cause turbidity in GA solutions of 5-6 mg/mL, both at 5° C. and 25° C./60% relative humidity storage.

A special study was performed in order to explain the reason for gelation of a great number of various formulations containing both actives—GA and laquinimod. Results of the study showed that gel formation was attributable to:
1. Reassembly of the GA polypeptides in the presence of laquinimod molecules, accompanied by appearance of hydrophobic nuclei and formation of fibrous structures through non-covalent forces, forming a three-dimensional gel network.
2. The hydrophobic nature of the interaction between GA and laquinimod.
3. Tyrosine residues playing a key role in the gel formation process.
4. The solution and GA-LAQ supernatant solution adopt an α-helical structure, while the sedimented gel-like fraction of GA-LAQ has the more ordered β-sheet conformation.

The study also showed that kinetics of the gelation of GA-LAQ strongly correlated with elevation of temperature, prolongation of incubation time and, in particular, with the concentration of LAQ in the tested mixtures.

Surprisingly, as disclosed herein, the inclusion of amino acids in formulations of laquinimod and GA eliminates gelation of the final product.

Good manufacturing results of a liquid clear fixed-dose GA-laquinimod formulation with no gel formation with their maximal possible concentrations—about 30 mg/mL of GA and 1-1.3 mg/mL Laq-Na—were achieved using amino acids (lysine, proline, alanine, glycine, histidine). The process is highly reproducible and technology permits industrial manufacturing of the clear and stable drug product involving sterile filtration stages. Formulations with lysine or alanine did not undergo gel formation during storage at 2-8° C. for more than two years. Similar results were obtained with other amino acids.

### REFERENCES

1. Filippi et al., Glatiramer acetate reduces the proportion of MS lesions evolving into black holes, Neurol., 2001, 57:731-733.
2. Johnson et al., Copolymer 1 reduces relapse rate and improves disability in relapsing-remitting multiple sclerosis: results of a phase III multicenter, double-blind placebo-controlled trial. The Copolymer 1 Multiple Sclerosis Study Group, Neurol., 1995, 45:1268.
3. Lampert, Autoimmune and virus-induced demyelinating diseases. A review, Am. J. Path., 1978, 91:176-208.
4. PCT International Application Publication No. WO 2005/074899, published Aug. 18, 2005 (ACTIVE BIOTECH AB).
5. PCT International Application Publication No. WO 2007/047863, published Apr. 26, 2007 (TEVA PHARMACEUTICAL INDUSTRIES, LTD.).
6. PCT International Application Publication No. WO/2007/146248, published Dec. 21, 2007 (TEVA PHARMACEUTICAL INDUSTRIES, LTD.).
7. PCT International Application Publication No. WO 1998/30227, published Jul. 16, 1998.
8. PCT International Application Publication No. WO 2000/05250, published Feb. 3, 2000.
9. PCT International Application Publication No. WO 2000/18794, published Apr. 6, 2000.
10. PCT International Application Publication No. WO 2003/048735, published Jun. 12, 2003.
11. PCT International Application Publication No. WO 2004/103297, published Dec. 2, 2004.
12. PCT International Application Publication No. WO 2006/029393, published Mar. 16, 2006.
13. PCT International Application Publication No. WO 2006/029411, published Mar. 16, 2006.
14. PCT International Application Publication No. WO 2006/083608, published Aug. 10, 2006.
15. PCT International Application Publication No. WO 2006/089164, published Aug. 24, 2006.
16. PCT International Application Publication No. WO 2006/116602, published Nov. 2, 2006.
17. PCT International Application Publication No. WO 2009/070298, published Jun. 4, 2009.
18. PCT International Application Publication No. WO 2011/008274, published Jan. 20, 2011.
19. PCT International Application Publication No. WO 2011/022063, published Feb. 24, 2011.
20. PCT International Application Publication No. WO 2012/051106, published Apr. 19, 2012.
21. Polman et al., (2005) “Treatment with laquinimod reduces development of active MRI lesions in relapsing MS”, Neurology. 64:987-991.
22. Sandberg-Wollheim et al., (2005) “48-week open safety study with high-dose oral laquinimod in patients”, Mult Scler. 11:5154.
23. U.S. Patent Application Publication No. 2005/0192315, published Sep. 1, 2005 (Jansson et al.).
24. U.S. Patent Application Publication No. 2013/0029916, published Jan. 31, 2013 (Gilgun and Tarcic).
25. U.S. Patent Application Publication No. 2008-0207526, published Aug. 28, 2008 (Strominger et al.).
26. U.S. Patent Application Publication No. 2011-0230413, published Sep. 22, 2011 (Suhayl Dhib-Jalbut).
27. U.S. Patent Application Publication No. 2012-0010238, published Jan. 12, 2012 (Fristedt).
28. U.S. Patent Application Publication No. 2012-0010239, published Jan. 12, 2012 (Piryatinsky et al.).
29. U.S. Patent Application Publication No. 2010-0055072, published Mar. 4, 2010 (Gant et al.).
30. U.S. Pat. No. 3,849,550, issued Nov. 19, 1974 (Teitelbaum et al).
31. U.S. Pat. No. 5,800,808, issued Sep. 1, 1998 (Konfino et al).
32. U.S. Pat. No. 5,858,964, issued Jan. 12, 1999 (Aharoni et al).
33. U.S. Pat. No. 5,981,589, issued Nov. 9, 1999 (Konfino et al).
34. U.S. Pat. No. 6,048,898, issued Apr. 11, 2000 (Konfino et al).
35. U.S. Pat. No. 6,054,430, issued Apr. 25, 2000 (Konfino et al).
36. U.S. Pat. No. 6,077,851, issued Jun. 20, 2000 (Bjork et al).
37. U.S. Pat. No. 6,214,791, issued Apr. 10, 2001 (Arnon et al).
38. U.S. Pat. No. 6,342,476, issued Jan. 29, 2002 (Konfino et al).
39. U.S. Pat. No. 6,362,161, issued Mar. 26, 2002 (Konfino et al).
40. U.S. Pat. No. 6,875,869, issued Apr. 5, 2005 (Jansson).
41. U.S. Pat. No. 7,556,767, issued Jun. 17, 2009 (Lin et al.).
42. U.S. Pat. No. 7,589,208, issued Sep. 15, 2009 (Jansson et al.).
43. U.S. Pat. No. 7,884,208, issued Feb. 8, 2011 (Frenkel et al.).
44. U.S. Pat. No. 7,989,473, issued Aug. 2, 2011 (Patashnik et al.).
45. U.S. Pat. No. 8,008,258, issued Aug. 10, 2011 (Aharoni et al.).
46. U.S. Pat. No. 8,178,127, issued May 15, 2012 (Muhammad Safadi et al.).
47. U.S. Pat. No. 8,399,413, issued Feb. 27, 2013 (Klinger).

1. A stable pharmaceutical composition comprising:
a) a therapeutically effective amount of laquinimod, a therapeutically effective amount of a glatiramer acetate related drug substance, preferably glatiramer acetate, and an amount of an amino acid; or
b) a therapeutically effective amount of laquinimod and an amount of an amino acid; or
c) a glatiramer acetate related drug substance, preferably glatiramer acetate, and an amount of an amino acid.

2-3. (canceled)

4. The composition of claim 1 in liquid form, and/or wherein the composition is an aqueous solution, and/or wherein the composition is clear, and/or wherein the composition is isotonic, or wherein the composition is optionally a lyophilized powder.

5-9. (canceled)

10. The composition of claim 1, wherein the laquinimod is potassium salt, lithium salt, sodium salt, calcium salt, or laquinimod sodium, or wherein the laquinimod is optionally laquinimod acid.

11-12. (canceled)

13. The composition of claim 1, wherein the therapeutically effective amount of laquinimod is less than 0.6 mg, 0.25 mg-1.5 mg, 0.1 mg, 0.25 mg, 0.3 mg, 0.5 mg, 0.6 mg, 1.0 mg, or 1.2 mg, and wherein the therapeutically effective amount of laquinimod has optionally a concentration of 0.7-3 mg/mL, 0.7 mg/mL, 0.8 mg/mL, 0.9 mg/mL, 1 mg/mL, 1.1 mg/mL, 1.25 mg/mL, 1.5 mg/mL, 2 mg/mL, or 3 mg/mL; and wherein the therapeutically effective amount of glatiramer acetate related drug substance is optionally 0.1-1000 mg, 50-150 mg, 0.1-70 mg, 10-80 mg, 1 mg, 5 mg, 15 mg, 20 mg, 30 mg, 40 mg, 50 mg, or 100 mg, and wherein the therapeutically effective amount of glatiramer acetate related drug substance has optionally a concentration of 5-40 mg/mL, 5 mg/mL, 10 mg/mL, 30 mg/mL, 35 mg/mL, or 40 mg/mL.

14-25. (canceled)

26. The composition of claim 1, wherein the composition is in a unit dose, comprising 0.1 mg, 0.25 mg, 0.3 mg, 0.5 mg, 0.6 mg, 1.0 mg, or 1.2 mg of laquinimod and 1 mg, 5 mg, 15 mg, 20 mg, 30 mg, 40 mg, 50 mg, or 100 mg of glatiramer acetate related drug substance, wherein the unit dose has optionally a volume of 0.5-5 mL, 1 mL, 1.5 mL, 2 mL, 3 mL, 4 mL, or 5 mL, and wherein the unit dose is optionally in a syringe, a vial, an ampule, a cartridge, or an infusion, or optionally a pre-filled syringe for administration.

27-31. (canceled)

32. The composition of claim 1, wherein the amino acid is selected from lysine, glycine, proline, alanine, or histidine, and preferably wherein the amino acid has a concentration of 0.5-22 mg/mL, more preferably 0.5-5 mg/mL, 0.5-3.5 mg/mL, 0.7-1.8 mg/mL, 0.5 mg/mL, 0.6 mg/mL, 0.7 mg/mL, 0.8 mg/mL, 0.9 mg/mL, 1 mg/mL, 1.1 mg/mL, 1.2 mg/mL, 1.3 mg/mL, 1.4 mg/mL, 1.5 mg/mL, 2 mg/mL, 2.25 mg/mL, 2.5 mg/mL, 3 mg/mL, 3.5 mg/mL, 4 mg/mL, or 5 mg/mL.

33-35. (canceled)

36. The composition of claim 1, wherein the amino acid is lysine, and wherein the concentration ratio of lysine to laquinimod (Lys:Laq) is optionally from about 0.4 mg/mL:1 mg/mL to about 3.2 mg/mL:1 mg/mL; or wherein the amino acid is optionally glycine, and wherein the concentration ratio of glycine to laquinimod (Gly:Laq) is optionally from about 0.5 mg/mL:1 mg/mL to about 4.1 mg/mL:1 mg/mL; or wherein the amino acid is optionally proline, and wherein the concentration ratio of proline to laquinimod (Pro:Laq) is optionally from about 1.5 mg/mL:1 mg/mL to about 2.2 mg/mL:1 mg/mL; or wherein the amino acid is optionally alanine, and wherein the concentration ratio of alanine to laquinimod (Ala:Laq) is optionally from about 0.6 mg/mL:1 mg/mL to about 0.7 mg/mL:1 mg/mL; or wherein the amino acid is optionally histidine, and wherein the concentration ratio of histidine to laquinimod (His:Laq) is optionally from about 1.2 mg/mL:1 mg/mL to about 2.6 mg/mL:1 mg/mL.

37-45. (canceled)

46. The composition of claim 1, wherein the pharmaceutical composition further comprises a tonicity agent, wherein the tonicity agent is optionally mannitol, sodium chloride, or trehalose; and wherein mannitol has optionally a concentration of 7-50 mg/mL, 7 mg/mL, 20 mg/mL, 25 mg/mL, 30 mg/mL, 35 mg/mL, 37.5 mg/mL, 40 mg/mL, 43 mg/mL, 45 mg/mL, or 50 mg/mL; and wherein sodium chloride has optionally a concentration of 5-10 mg/mL, 5 mg/mL, 6 mg/mL, 7 mg/mL, 8 mg/mL, 9 mg/mL, or 10 mg/mL; and wherein the pharmaceutical composition further comprises optionally a buffer having a concentration of 0.1-0.5 mol/L, 0.1 mol/L, or 0.5 mol/L, wherein the buffer is optionally histidine, phosphate-buffered saline, phosphate salt, hydrocarbonate, or acetate, and wherein the phosphate salt is optionally a sodium salt, potassium salt, or sodium-potassium salt, the hydrocarbonate is optionally sodium bicarbonate, and the acetate is optionally sodium acetate.

47-60. (canceled)

61. The composition of claim 1, wherein the composition is prepared for periodic administration, wherein the periodic administration continues optionally for more than 30 days, for more than 42 days, or for 6 months or more; and wherein the composition is optionally prepared for administration once daily, more often than once daily, less often than once daily, or three times per week; and wherein the composition is optionally prepared for administration by subcutaneous injection or through an intravenous, intraperitoneal, intramuscular, intranasal, buccal, vaginal, rectal, intraocular, intrathecal, topical, oral, or intradermal route.

62-68. (canceled)

69. A process for making the stable pharmaceutical composition comprising a therapeutically effective amount of laquinimod, a therapeutically effective amount of glatiramer acetate related drug substance, preferably glatiramer acetate, and an amount of an amino acid of claim 1 in liquid form comprising: a) obtaining the laquinimod, the glatiramer acetate related drug substance, preferably glatiramer acetate, and the amino acid, and b) forming the liquid form of the laquinimod, the glatiramer acetate related drug substance, preferably glatiramer acetate, and the amino acid.

70. A process for making the stable pharmaceutical composition comprising a therapeutically effective amount of glatiramer acetate related drug substance, preferably glatiramer acetate, and an amount of an amino acid of claim 1 in liquid form comprising: a) obtaining the glatiramer acetate related drug substance, preferably glatiramer acetate, and the amino acid, and b) forming the liquid form of the glatiramer acetate related drug substance, preferably glatiramer acetate, and the amino acid.

71. (canceled)

72. A process for making the stable pharmaceutical composition comprising a therapeutically effective amount of laquinimod and an amount of an amino acid of claim 1 in liquid form comprising: a) obtaining the laquinimod and the amino acid, and b) forming the liquid form of the laquinimod and the amino acid.

73. The process of claim 69, wherein the process comprises forming a liquid form of the glatiramer acetate related drug substance, preferably glatiramer acetate, and the amino acid prior to forming the liquid form of the laquinimod, the glatiramer acetate related drug substance, preferably glatiramer acetate, and the amino acid; or wherein the process comprises forming a liquid form of the laquinimod and the amino acid prior to forming the liquid form of the laquinimod, the glatiramer acetate related drug substance, preferably glatiramer acetate, and the amino acid.

74. (canceled)

75. The process of claim 70, wherein the process comprises forming a liquid form of the glatiramer acetate related drug substance, preferably glatiramer acetate, prior to forming the liquid form of the glatiramer acetate related drug substance, preferably glatiramer acetate, and the amino acid; or wherein the process comprises forming a liquid form of the amino acid prior to forming the liquid form of the glatiramer acetate related drug substance, preferably glatiramer acetate, and the amino acid.

76. (canceled)

77. The process of claim 72, wherein the process comprises forming a liquid form of the laquinimod prior to forming the liquid form of the laquinimod and the amino acid; or wherein the process comprises forming a liquid form of the amino acid prior to forming the liquid form of the laquinimod and the amino acid.

78. (canceled)

79. The process of claim 69, wherein the liquid form is formed at stirring, is clear, and/or is an aqueous solution.

80. The process of claim 73, wherein the process comprises passing the liquid form through a cellulose acetate syringe filter prior to forming a liquid form of the laquinimod, the glatiramer acetate related drug substance, preferably glatiramer acetate, and the amino acid; or wherein the process comprises storing the liquid form prior to forming a liquid form of the laquinimod, the glatiramer acetate related drug substance, and the amino acid, wherein the liquid form is stored at 2-8° C.

81-84. (canceled)

85. A stable pharmaceutical composition comprising a therapeutically effective amount of laquinimod, a therapeutically effective amount of a glatiramer acetate related drug substance, preferably glatiramer acetate, and an amount of an amino acid, prepared by the process of claim 69.

86. (canceled)

87. A sealed package comprising the composition of claim 1, wherein the sealed package optionally after storage at 25° C. and at a relative humidity (RH) of 60% for at least 6 months or at least 12 months is free of gelation, wherein gelation is optionally evaluated using the Ubbelohde tube method, and wherein the sealed package is optionally a syringe, a vial, an ampule, a cartridge, or an infusion, or optionally a pre-filled syringe for administration.

88-93. (canceled)

94. A method for i) treating a subject afflicted with a form of autoimmune disease, and/or ii) treating a subject afflicted with multiple sclerosis or presenting a clinically isolated syndrome, and/or iii) alleviating a symptom of multiple sclerosis in a subject afflicted with multiple sclerosis or presenting a clinically isolated syndrome, and/or iv) providing neuroprotection to a subject in need thereof, comprising administering to the subject the composition of claim 1 so as to thereby treat and/or alleviate the symptom of multiple sclerosis in the subject and/or provide neuroprotection to a subject in need thereof.

95-102. (canceled)

103. A pharmaceutical oral unit dosage form of a therapeutically effective amount of laquinimod, a therapeutically effective amount of glatiramer acetate related drug substance, and an amount of an amino acid.

104. A package comprising:
a) one or more unit doses, each such unit dose comprising:
i) a therapeutically effective amount of laquinimod,
ii) a therapeutically effective amount of glatiramer acetate related drug substance, and
iii) an amount of an amino acid, and
b) instructions for use of the one or more unit doses for
i) treating a subject afflicted with an autoimmune disease,
ii) treating a subject afflicted with multiple sclerosis or presenting a clinically isolated syndrome, and/or alleviating a symptom of multiple sclerosis in a subject afflicted with multiple sclerosis or presenting a clinically isolated syndrome, and/or
iii) providing neuroprotection to a subject in need thereof.
